(19) United States
(12) Patent Application Publication
(10) Pub. No.: US 2007/0225585 A1
(43) Pub. Date: Sep. 27, 2007

(54) HEADSET FOR ELECTRODES

(76) Inventors: Lori Ann Washbon, San Francisco, CA (US); Emir Delic, Lidcombe (AU)

Correspondence Address: FISH & RICHARDSON P.C., PO BOX 1022, MINNEAPOLIS, MN, 55440-1022, US

(21) Appl. No.: 11/689,127

(22) Filed: Mar. 21, 2007

Related U.S. Application Data
(60) Provisional application No. 60/743,641, filed on Mar. 22, 2006.
(60) Provisional application No. 60/868,927, filed on Dec. 6, 2006.

(51) Int. Cl. A61B 5/04

(52) U.S. Cl. 600/393; 600/383

(57) ABSTRACT

An electrode headset in which electrodes can be mounted is described. The electrode headset can be formed from substantially rigid components including some flexibility to snugly embrace a variety of head shapes and sizes, while providing reliable positioning of electrodes according to an electrode placement scheme on a subject's head. In another implementation, the electrode headset can be formed from soft and stretchable bands with voids created therebetween.

### CROSS REFERENCE TO RELATED APPLICATIONS

This application claims priority to pending U.S. Provisional Application Ser. No. 60/743,641, entitled “Capturing Bioelectric Signals”, filed on Mar. 22, 2006, and to pending U.S. Provisional Application Ser. No. 60/868,927, entitled “Electrode and Electrode Headset”, filed on Dec. 6, 2006, and the entire contents of both applications hereby incorporated by reference.

### TECHNICAL FIELD

This invention relates to an apparatus that can include mounts for one or more electrodes.

### BACKGROUND

An electrode system to capture bioelectric signals, such as electroencephalograph (EEG) signals, from a subject generally should address various requirements including safety needs, cost, power consumption, performance, ease of use and subject comfort. In a non-clinical application the relative importance of these factors may be somewhat different to that in a clinical application. For example, in a clinical application the electrodes are applied by a relatively skilled technician, whereas in non-clinical application the electrodes are more likely to be applied by a person with no training or knowledge of correct application or placement of the electrodes. Convenience and subject comfort are also generally more important in a non-clinical application. A patient in a clinical situation is more likely to be tolerant of some level of discomfort or inconvenience when testing and calibrating electrodes than a person in a non-clinical setting.

Conventional electrodes include passive electrodes and active electrodes. Passive electrodes follow a simple design principle and include a metal disc with a connecting wire to electronic circuitry. The simplicity makes this type of electrode low cost, although these electrodes are prone to noise and can require numerous noise canceling techniques to achieve satisfactory performance. One noise canceling technique, to minimize impedance at the skin-electrode interface and to minimize interference, involves conditioning the skin where the electrode is to be applied. Typically a scalpel is used to scrape the skin and a liquid disinfectant solution is used to clean the area. Another approach to minimizing impedance and interference at the skin-electrode interface is to fill any gap at the interface with a conductive gel or saline solution that can regulate the impedance.

Active electrodes include resistive and capacitive active electrodes. Resistive active electrodes use a direct current path between the subject's skin and the input of an operational amplifier to acquire a signal. Capacitive active electrodes do not make electrical contact with the subject's skin, but have a capacitive link between subject's skin and the electrode.

Active electrodes apply the principle of impedance transformation at the electrode site to improve signal acquisition performance. The electrode plate can be connected to a buffer circuit made from a high input impedance op-amp. The large input impedance of the op-amp can make the impedance at the skin-electrode interface insignificant and stabilize the skin-electrode interface, resulting in improved recording even without use of gel or saline solution. The addition of gel or saline can improve performance even more over passive electrodes. Another advantage of active over passive electrodes is that the impedance of wires connecting active electrodes to an acquisition device can be close to zero, effectively combating common mode and power line interference that can be introduced at this stage. However, the improvements in performance come at the expense of price, as active electrodes require at least one op-amp per electrode, increased power consumption and introduce the need for extra wires to deliver power to the active electrodes. Additionally, because active electrodes are more sensitive than passive electrodes, they can be extremely sensitive to movement, adding artifacts into the acquired signal. Thus, care is needed to ensure firm and stable contact between active electrodes and the skin. If active electrodes are used without a gel or saline solution, it can be difficult to get successful performance, particularly at locations on the head covered with hair.

Capacitive active electrodes are a fairly recent development in EEG signal acquisition. These electrodes do not require electrical contact to be made between the subject and the electrode plate to acquire a signal. The electrode plate can be maintained a predetermined distance away from the head by a highly dielectric material and signals are then detected via fluctuations in capacitance.

A conventional apparatus for applying electrodes to a subject's head includes a flexible cap that covers the subject's entire scalp and includes a strap beneath the chin, so that the cap may be snugly secured to the subject's head. This type of apparatus is typically used in a clinical setting and can include over 100 electrodes for some applications.

### SUMMARY

This invention relates to an apparatus that can be used to mount one or more bio-sensors and can include the bio-sensors. In one implementation, the bio-sensors are electrodes. In general, in one aspect, the invention features an electrode headset including multiple rigid bands and one or more electrode mounts included within the rigid bands. The rigid bands are formed from a material including at least enough flexibility to flex in response to the electrode headset being positioned on a subject's head, such that the rigid bands embrace the subject's head. Each electrode mount is configured to mount an electrode and when the electrode headset is positioned on the subject's head, one or more electrodes mounted therein are positioned according to a desired electrode placement scheme relative to the subject's head.

Implementations of the invention can include one or more of the following features. The electrode headset can further include multiple voids formed between the rigid bands, such that portions of the top of the subject's head are exposed when the subject is wearing the electrode headset. Each of the one or more electrode mounts can be configured as an aperture formed within one of the rigid bands. The aperture is configured to receive and securely hold an electrode in position against the subject's head. The electrode headset can further include electronic circuitry configured to electrically connect to each of the one or more electrodes mounted within the electrode mounts, receive signals from the one or more electrodes and provide an output signal to a processor. The electronic circuitry can be embedded within, or mounted on, one of the plurality of rigid bands.

In general, in another aspect, the invention features an electrode headset including rigid bands and one or more electrode mounts included within the rigid bands. The rigid bands are formed from a material with sufficient flexibility to flex in response to the electrode headset being positioned on a subject's head. However, the material also exhibits sufficient resilience, such that when the electrode headset is positioned on the subject's head and electrodes are mounted in the electrode mounts, the rigid bands press the electrodes against the subject's head.

Implementations of the invention can include one or more of the following features. The rigid bands can include a first band to position on a first side of the subject's head and a second band to position on a second side of the subject's head, where the headset is configured to urge the first band and the second band toward each other. In one implementation, the first band can be a left temporal band to extend from a back portion of the subject's head toward the subject's left temporal region, and the second band can be a right temporal band to extend from a back portion of the subject's head toward the subject's right temporal region. Another implementation can further include a center band to position along a central back portion of the subject's head, and each of the first and second bands can be a dorsal band extending from the back portion of the subject's head toward opposite sides of the subject's forehead. Voids can be formed between the rigid bands, such that portions of the top of the subject's head are exposed when the subject is wearing the electrode headset. Each of the one or more electrode mounts can be an aperture formed within one of the rigid bands, where the aperture is configured to receive and securely hold an electrode in position against the subject's head. The electrode headset can further include electronic circuitry configured to electrically connect to each of the one or more electrodes mounted within the electrode mounts, receive signals from the one or more electrodes, and provide an output signal to a processor. The electronic circuitry can be embedded within or mounted on one of the plurality of rigid bands.

In general, in another aspect, the invention features an electrode headset including multiple rigid bands including a center band, left and right dorsal bands and left and right temporal bands. The center band is configured to position along a central back portion of a subject's head. Each dorsal band projects from the center band and extends from the back portion of the subject's head toward the subject's forehead. Each temporal band projects from the center band and extends from the back portion of the subject's head toward the subject's left and right temporal regions respectively. The electrode headset further includes electrode mounts formed within the rigid bands. Each electrode mount is configured to receive and mount an electrode and when the electrode headset is positioned on the subject's head, electrodes mounted therein are positioned according to a desired electrode placement scheme relative to the subject's head.

In general, in another aspect, the invention features an electrode headset including multiple rigid bands including a center band and left and right temporal bands. The center band is configured to position along a central back portion of a subject's head. The center band includes a middle portion positioned between a lower portion extending toward the lower rear of the subject's head and a forked upper portion extending toward the upper back of the subject's head and substantially symmetrical about a sagittal plane. Each temporal band projects from the center band and extends from the rear of the subject's head toward the subject's left and right temporal regions respectively. Each temporal band includes at least a first and a second finger extending from the distal end of the temporal band. The first and second fingers include distal ends terminating in the frontal region of the subject's head. The electrode headset further includes electrode mounts formed within each of the center, left temporal and right temporal bands. Each electrode mount is configured to receive and mount an electrode and when the electrode headset is positioned on the subject's head, one or more electrodes mounted therein are positioned according to a desired electrode placement scheme relative to the subject's head.

Implementations of the invention can include one or more of the following features. The second finger of each of the left and right temporal bands is forked at the distal end providing two distal termination points, where each distal termination point includes an electrode mount. The electrode headset can further include a chin strap extending from the left temporal band to the right temporal band, and configured to position beneath a chin of the subject during use.

In general, in another aspect, the invention features an electrode headset including multiple bands formed from a soft and stretchable material conformable to a subject's head, such that the bands embrace the subject's head. Voids are formed between the bands, such that portions of the top of the subject's head remain exposed when the subject is wearing the electrode headset. The electrode headset further includes one or more electrode mounts within the plurality of bands. Each electrode mount is configured to mount an electrode, and when the electrode headset is positioned on the subject's head, one or more electrodes mounted therein are positioned according to a desired electrode placement scheme relative to the subject's head.

Implementations of the invention can include one or more of the following features. Each of the one or more electrode mounts can be an aperture formed within one of the bands and configured to receive and securely hold an electrode in position against the subject's head. In another implementation, each of the one or more electrode mounts can be a pocket formed within one of the bands. The pocket can be configured to receive and securely hold an electrode in position against the subject's head. The electrode headset can further include electronic circuitry configured to electrically connect to each of the one or more electrodes mounted within the electrode mounts, receive signals from the one or more electrodes, and provide an output signal to a processor.

Implementations of the invention can realize one or more of the following advantages. The electrode headset described herein can provide suitable electrode placement in an easy to don apparatus. A subject who is untrained as to electrode placement can easily use the electrode headset without the assistance of a trained technician. The electrode headset can apply the necessary pressure to sufficiently press each electrode to the subject's scalp to provide a suitably strong and clear signal, yet is comfortable for the subject wearing the headset. The configuration not only ensures that the electrodes mounted therein are properly positioned relative to the subject's head and in accordance with a desired electrode placement scheme, but can ensure that the electrodes will remain in a substantially stable position throughout use. The good contact provided at the electrode-scalp interface can allow noise to settle relatively quickly, and a clean signal can be achieved relatively quickly as compared to prior art systems.

The electrode mounts are configured to allow individual electrodes to be easily mounted or replaced, independent of other electrodes mounted within the headset. Accordingly, if a single electrode malfunctions, the individual electrode can be replaced, rather than having to discard the entire electrode headset including all electrodes mounted therein. Additionally, the headset is configured to accommodate a range of head shapes and sizes.

The electrodes described herein are particularly suitable to a non-clinical application, where the subject's comfort and ease of use are important factors, although they can be used in a clinical application as well. The embodiments of dry electrodes described are advantageous for using the electrode headsets described herein, as they can provide a strong and clear signal even through a subject's hair and without use of a wetting fluid. The gimbaled contact can allow a suitable contact to be maintained at the electrode-subject interface, while permitting some relative movement between the electrode headset and the subject's head. The embodiments of wet electrodes described are also suitable for use with the electrode headsets described herein. The wetted conductive pad works well with a subject's hair and leaves the hair only slightly damp upon removal of the electrodes.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic representation of a signal acquisition system.

FIG. 2 is a schematic representation of a 10-20 electrode placement system.

FIGS. 3A-G show an implementation of a rigid electrode headset.

FIG. 4 is a schematic representation of an electrode placement scheme.

FIGS. 5A-B show an alternative implementation of a rigid electrode headset.

FIGS. 6A-C show an alternative implementation of a rigid electrode headset.

FIGS. 7A-B show an implementation of a soft electrode headset.

FIG. 8 shows an implementation of an electrode mount configured as a pocket.

FIGS. 9A-E show an implementation of an electrode.

FIGS. 10A-C show alternative implementations of contact elements included in the electrode shown in FIGS. 9A-E.

FIGS. 11A-B show an alternative electrode.

FIGS. 12A-B show an alternative electrode.

FIG. 13 is a schematic representation of a circuit diagram.

FIGS. 14A-B show an implementation of an electrode housing.

Like reference symbols in the various drawings indicate like elements.

### DETAILED DESCRIPTION

An electrode headset configured to position one or more electrodes mounted in the headset within a predetermined target region on a subject's head and in accordance with a desired electrode placement scheme is described. In one implementation, the electrode headset is formed from a hard material. That is, the electrode headset is formed from a substantially rigid material including at least some flexibility so as to comfortably embrace the subject's head, while applying sufficient pressure between the one more electrodes mounted therein and the subject's head. The one or more electrodes can be configured as a dry electrode or a wet electrode, where a dry electrode can obtain a signal without a conductive and typically wet material between the electrode and the subject's skin, and a wet material does require such a conductive material. The electrode headset does not cover the entire upper surface of the subject's head, and can be configured to reduce the region of the head in contact with the electrode headset, while being sufficiently comfortable and acceptable in non-clinical environment.

In another implementation, the electrode headset is formed from a soft and stretchable material. The soft electrode headset also does not cover the entire upper surface of the subject's head. The soft electrode headset is configured to fit snugly on the subject's head so as to apply sufficient pressure between the one or more electrodes mounted therein and the subject's head. The stretchable material has sufficient resilience to tend to embrace the subject's head.

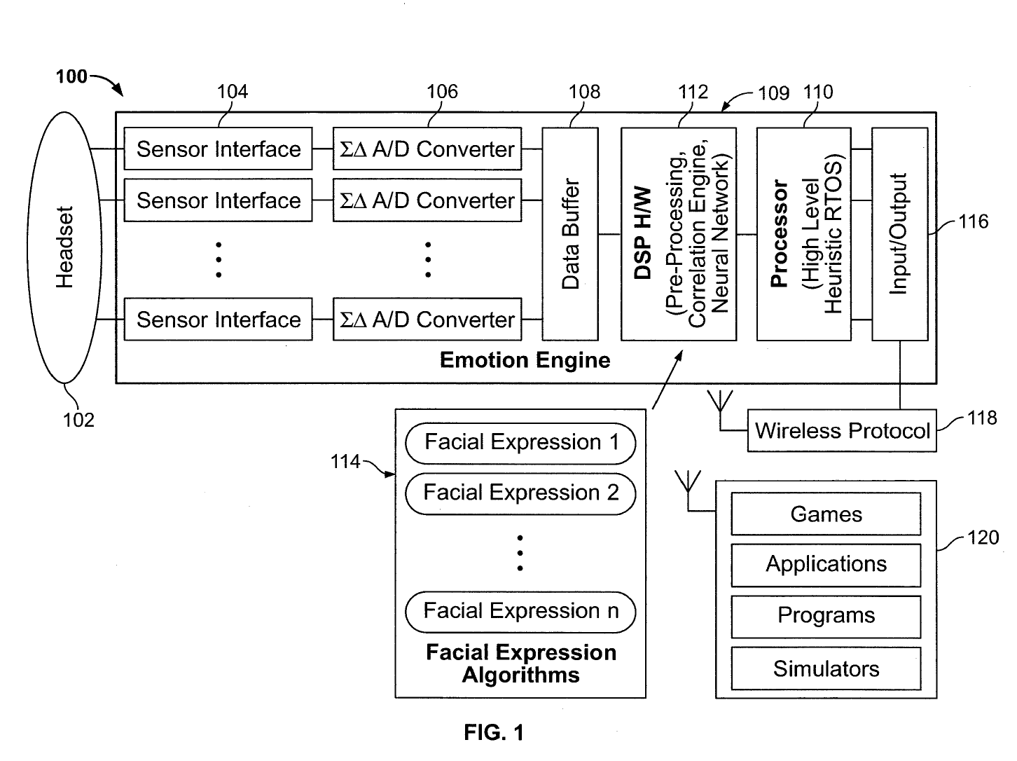

FIG. 1 is a schematic representation of a system for detecting and classifying mental states. The system is one example of a system that can employ the electrode headset and/or electrodes described herein. It should be understood however that other systems can use the headset and electrodes described, and the system shown in FIG. 1 is but one implementation for illustrative purposes.

The system includes a headset 102 configured to position one or more electrodes on a subject's head. The system is configured to operate generally as described in U.S. patent application Ser. No. 11/531,238, filed Sep. 12, 9006, entitled “Method and System for Detecting and Classifying the Mental State of a Subject”, and U.S. patent application Ser. No. 11/531,265, filed Sep. 12, 9006, entitled “Detection Of And Interaction Using Mental States”, both assigned to Emotiv Systems Pty Ltd, and which are hereby incorporated in their entirety by reference herein.

In one implementation, the one or more electrodes include signal acquisition electrodes configured to detect signals such as electroencephalograph (EEG) signals, electro-oculograph (EOG) signals, or similar electrical potentials in the body. Signals detected by the electrodes in the headset 102 are fed through a sensor interface 104 and digitized by an analog to digital converter 106. Digitized samples of the signal captured by each of the electrodes can be stored during operation of the system 100 in a data buffer 108 for subsequent processing.

The system 100 further includes a processing system 109 including a digital signal processor 112, a co-processing device 110 and associated memory for storing a series of instructions, otherwise known as a computer program or a computer control logic, to cause the processing system 109 to perform desired functional steps. Notably, the memory includes a series of instructions defining at least one algorithm 114 for detecting and classifying a predetermined type of mental state. Mental states determined by such a classification can include, but are not limited to: an emotion; a desire, an intention or conscious effort to perform an action such as performing an interaction with a real or virtual object; and a mental state corresponding to an actual movement made by the subject, such as a facial expression, blink, gesture etc. Upon detection of each predefined type of mental state, a corresponding control signal is transmitted to an input/output interface 116. From the input/output interface, the control sign can be transmitted via a wireless transmission device 118 or a wired link (not shown) to a platform 120 for use as a control input by a gaming application, program, simulator or other application.

In this embodiment, the processing of signals, e.g. the detection or classification of mental states is performed in software and the series of instructions is stored in the memory. In another embodiment, signal processing can be implemented primarily in hardware using, for example, hardware components such as an Application Specific Integrated Circuit (ASIC). Implementation of the hardware state machine so as to perform these functions will be apparent to persons skilled in the relevant art. In yet other embodiments signal processing can be implemented using a combination of both software and hardware.

In this embodiment the processing system 109 is arranged as separate to the platform 120, however the system 100 can be arranged in a variety of configurations that split the signal processing functionality between various groups of hardware, for example in some embodiments, at least part of the signal processing functionality can be implemented in electronics mounted on the headset 102 or in the platform 120. For example, the apparatus can include a headset assembly that includes the headset, a MUX, A/D converter(s) before or after the MUX, a wireless transmission device, a battery for power supply, and a microcontroller to control battery use, send data from the MUX or A/D converter to the wireless chip, and the like. The apparatus can also include a separate processor unit that includes a wireless receiver to receive data from the headset assembly, and the processing system, e.g., the digital signal processor and the co-processor. The processor unit can be connected to the platform by a wired or wireless connection. As another example, the apparatus can include a head set assembly as described above, the platform can include a wireless receiver to receive data from the headset assembly, and a digital signal processor dedicated to detection of mental states can be integrated directly into the platform. As yet another example, the apparatus can include a head set assembly as described above, the platform can include a wireless receiver to receive data from the headset assembly, and the mental state detection algorithms are performed in the platform by the same processor, e.g., a general purpose digital processor, that executes the application, programs, simulators or the like.

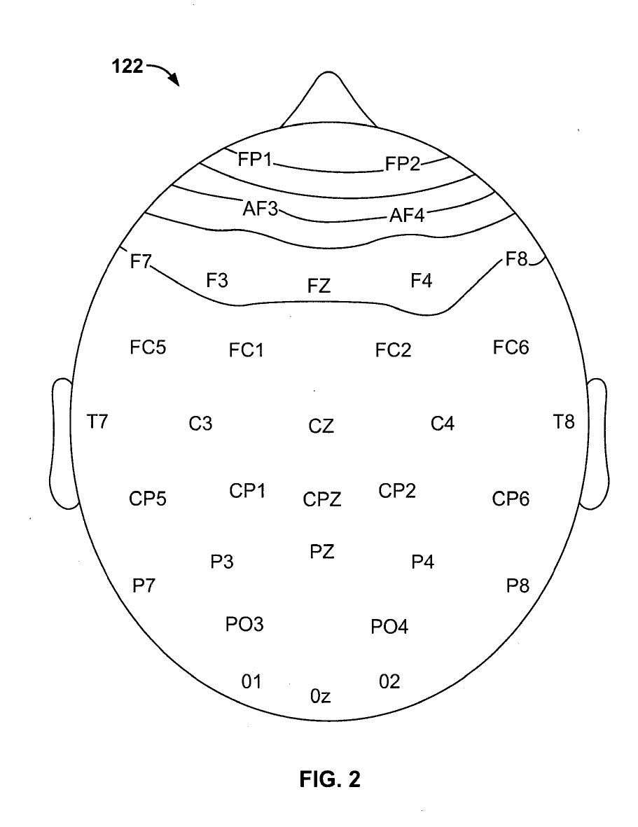

FIG. 2 shows a scheme 122 of electrode placement corresponding to the international 10-20 electrode placement system (the “10-20 system”). The 10-20 system is based on the relationship between the location of an electrode and the underlying area of cerebral cortex. Each point on the electrode placement scheme 122 indicates a possible scalp electrode position. Each position is indicated by a letter to identify a brain lobe and a number or other letter to identify a hemisphere location. The letters F, T, C, P, and O stand for the frontal, temporal, central, parietal and occipital lobes of the brain. Even numbers refer to the right hemisphere and odd numbers refer to the left hemisphere. The letter Z refers to an electrode placed on the mid-line. The mid-line is a line along the scalp on the sagittal plane originating at the nasion and ending at the inion at the back of the head. The “10” and “20” refer to percentages of the mid-line division. The mid-line is divided into 7 positions, namely, Nasion, Fpz, Fz, Cz, Pz, Oz and Inion, and the angular intervals between adjacent positions are set at 10%, 20%, 20%, 20%, 20% and 10% of the mid-line length respectively.

Rigid Electrode Headset

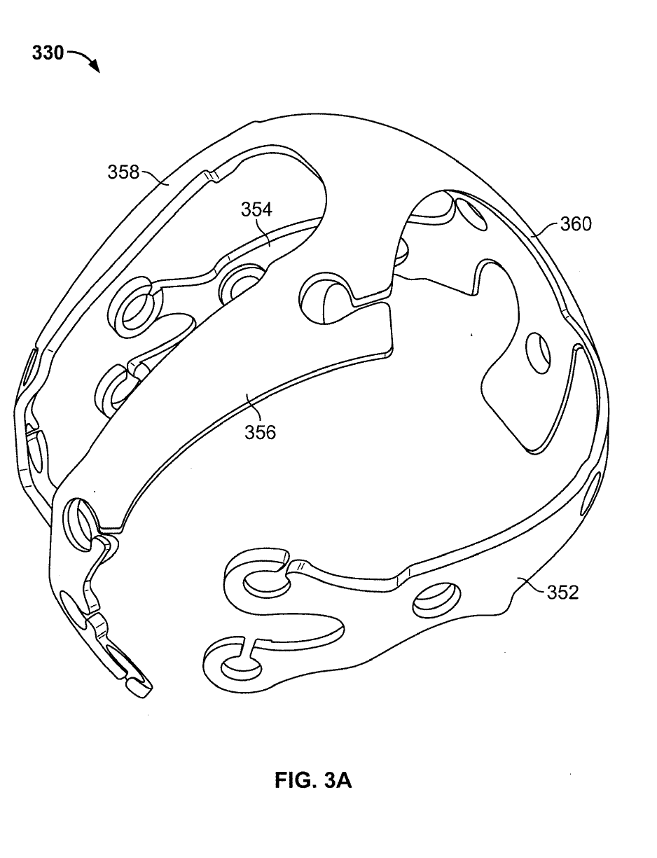

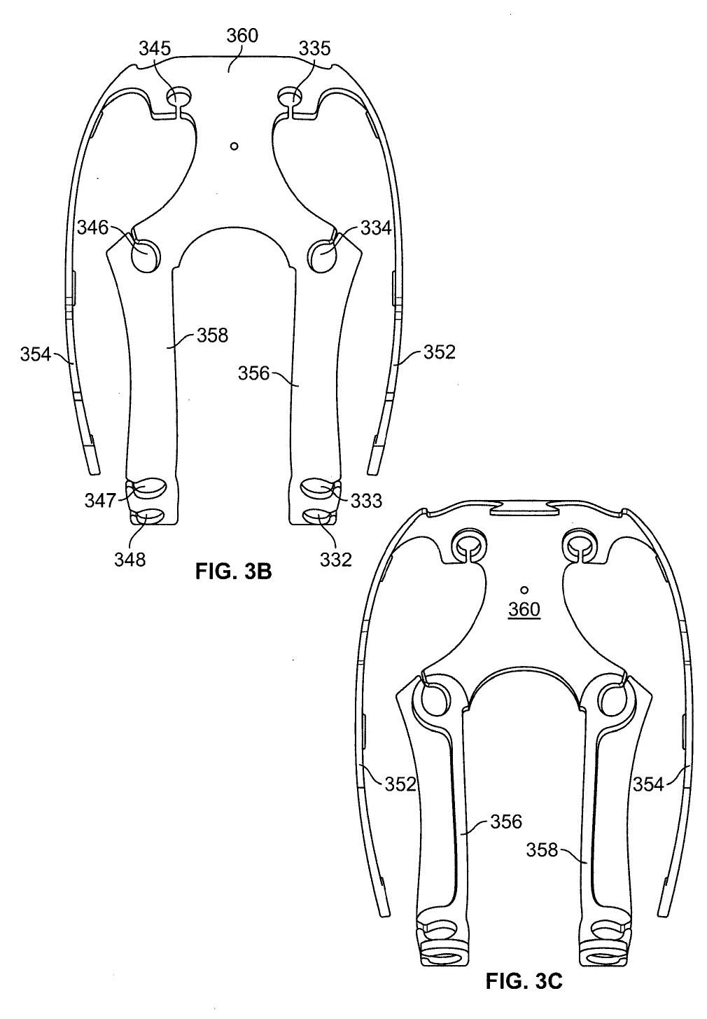

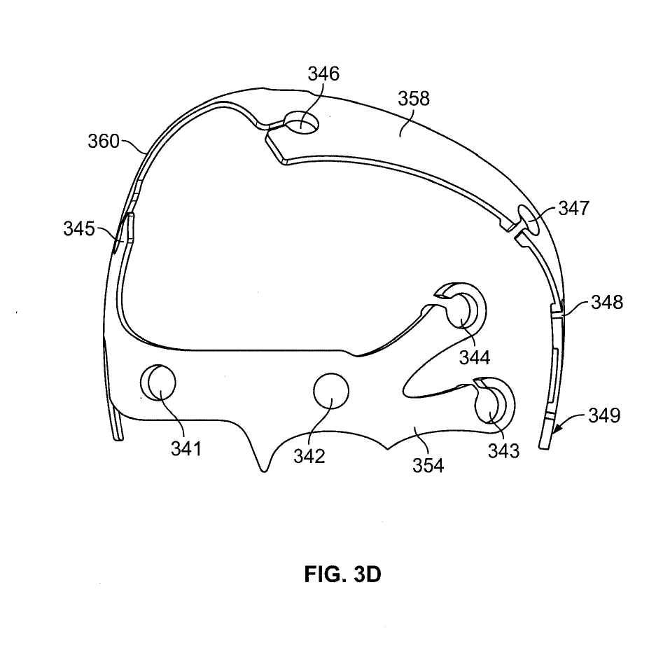

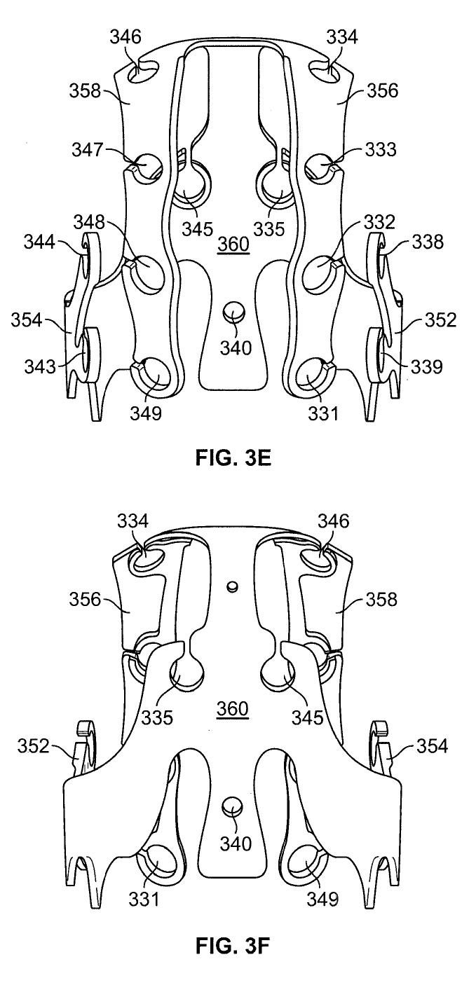

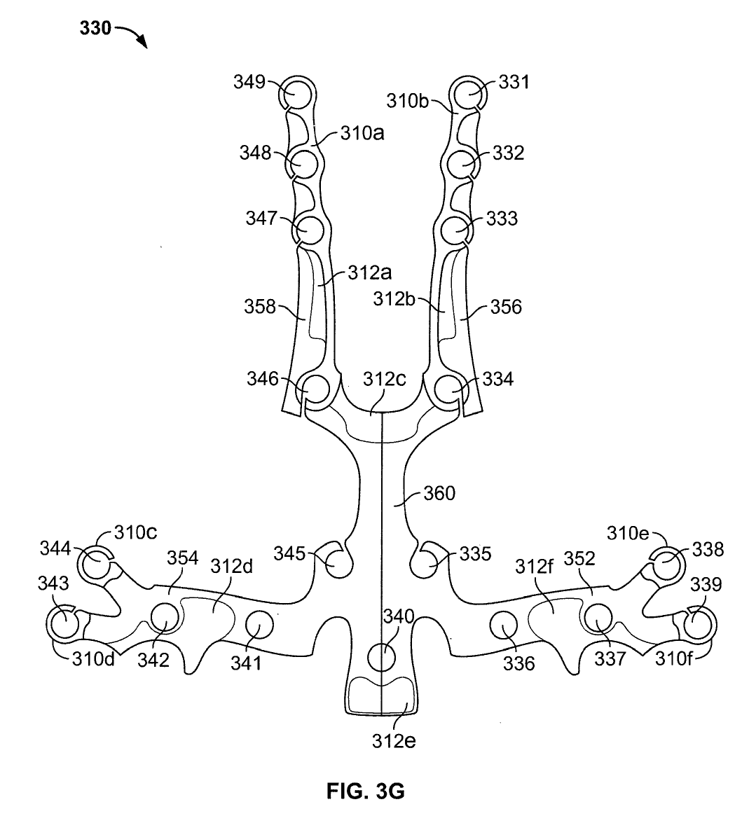

Referring to FIGS. 3A-F, various views of one implementation of an electrode headset 330 are shown. The electrode headset 330 is configured to fit snugly on a subject's head and can properly fit a range of head shapes and sizes. Multiple electrode mounts are included in the electrode headset 330 and are each configured to mount an electrode. In this implementation the electrode mounts are apertures configured to receive and mount an electrode therein, and shall be referred to as electrode apertures 331-349. However, it should be noted that other configurations of electrode mounts can be used. For example, an electrode can be mounted to the electrode headset using a clamp, screw or other suitable connection mechanism and/or configuration.

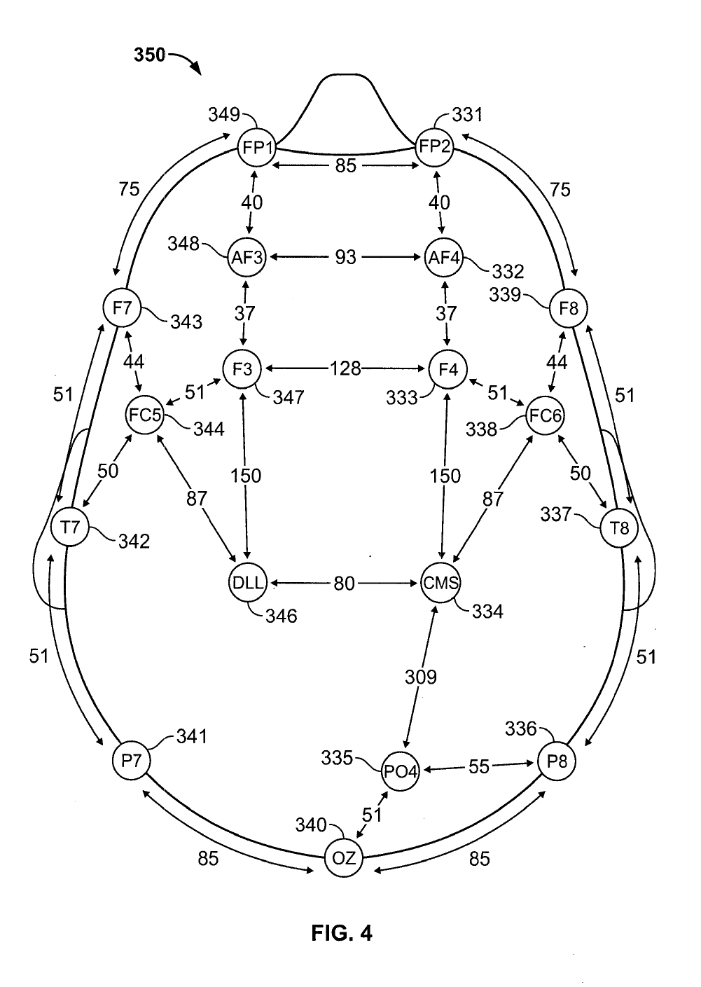

FIG. 3G shows an inner plan view of the electrode headset 330 illustrated as if the components were flattened out, providing a good view of the electrode aperture placement. In this particular implementation, the electrode headset 330 includes 19 electrode apertures 331-349 and can therefore mount 0 to 19 electrodes. Referring to FIG. 4, an electrode placement scheme 350 for a subset of the electrode placement positions included in the 10-20 system is shown. The subset of electrode placement positions corresponds to the electrode apertures 331-344 and 346-349 included in the electrode headset 330, and identify the target brain lobes for an electrode mounted within various of the electrode apertures. The numbering of the electrode apertures 331-344 and 346-349 is superimposed on the electrode placement scheme 350 shown in FIG. 4, to illustrate the correspondence between the electrode placement and the electrode apertures 331-349.

In the particular implementation of the electrode headset 330 shown, the electrode apertures 331-349 are positioned to mount electrodes to gather information about the subject's facial expression (i.e., facial muscle movement), emotions and cognitive information. The electrode headset 330 can be used with electrodes mounted in all or a subset of the electrode apertures 331-349. One or more apertures can be used to mount a reference electrode, i.e., an electrode to which signals received from other electrodes can be compared. In one implementation, the reference electrode can bias the subject's body to a known reference potential, e.g., one half of the analog supply voltage. Driven Right Leg (DRL) circuitry can compensate for external effects and keep the subject's body potential stable. The EEG signals can be referenced to the body potential supped by the reference electrode.

Referring again to FIGS. 3A-F, the structure of the implementation of the electrode headset 330 shall be described further. The electrode headset 330 includes a left temporal band 352, a right temporal band 354, a left dorsal band 356 and a right dorsal band 358. The bands 352-358 each connect to a center band 360. Each band is configured to provide one or more electrode apertures to a desired region on a subject's head when the electrode headset 330 is worn by the subject. Generally, to provide desired results a particular electrode must be placed within a region that is approximately twice the size of the target location, providing at least some leeway when positioning the electrode on the subject's head. Because some leeway is permissible, and because the electrode headset 330 is configured to conform to and embrace heads of various shapes and sizes, the electrode headset 330 can be used to accurately position in accordance with a desired electrode placement scheme a set of electrodes on a variety of head shapes with relative ease of use.

When the headset 330 is placed on a user's head, the center band 360 generally covers and contacts the posterior region of the user's scalp, extending upwardly along the parietal to near the top of the user's head. The left temporal band 352 and a right temporal band 354 extend from the center band 360 at the posterior region of the user's scalp, transversely around opposite sides of the head along the temple and toward the front of the head, ending before the orbits. The left dorsal band 356 and right dorsal band 358 extend from the center band near the top of the user's head, generally sagitally and in parallel along the frontal, ending above the orbits.

Without being limited to any particular theory, the electrode headset 330 may be able to fit a wide range of users because the compressive fit between the dorsal bands 356, 358 and the center band 360 provides a firm and stable attachment on the subject's head, permitting the temporal bands 352, 354 to flex to accommodate heads of different widths and shapes.

In this implementation, the left temporal band 352 includes four electrode apertures 341-344 and the corresponding right temporal band 354 includes four electrode apertures 336-339. The four electrodes that can be mounted on each temporal band are positioned to sense signals from the frontal, temporal, central and parietal lobes, as is shown in the electrode scheme 350 in FIG. 4. The temporal bands 352, 354 are formed from a substantially rigid material that includes some flexibility. The temporal bands 352, 354 in a base position (i.e., when not worn on a subject's head) are slightly curved toward the center of the electrode headset 330, as is shown clearly in the top view of FIG. 3B. When the subject places the electrode headset 330 upon the subject's head, the subject's head will urge the temporal bands 352, 354 away from the center. The flexibility in the temporal bands 352, 354 is sufficient to permit the subject's head to urge the temporal bands 352, 354 away from each other without breakage, yet rigid enough to maintain the overall shape of the temporal bands 352, 354. The temporal bands 352, 354 conform to and embrace the subject's head and provide a snug fit between the temporal bands 352, 354 and the subject's head.

As an example of the desired flexibility in the bands, the flexibility provided by polystyrene with an approximate thickness in the range of about 2-7 millimeters is suitable.

The snug fit between the temporal bands 352, 354, that is provided at least in part by the bands 352, 354 pressing against the subject's head in an effort to return to their base position, exerts sufficient pressure on the electrodes mounted within the electrode apertures 336-339 and 341-344 to provide contact at the electrode-subject interface suitable to obtain a sufficient signal.

In this implementation, the left dorsal band 356 and the right dorsal band 358 each include four electrode apertures 346-349 and 331-334 respectively. When the electrode headset 330 is worn by a subject, electrodes mounted within the electrode apertures 346-349 and 331-334 are positioned over the frontal lobes, as is shown in the electrode scheme 350 shown in FIG. 4. The acronyms DLL, DRL and CMS included in FIG. 4 stand for “Driven Left Leg”, “Driven Right Leg” and “Common Mode Signal” respectively. As is shown in FIG. 3D, when in a base position (i.e., not worn by a subject), the dorsal bands 356, 358 project horizontally with a downward slope and at their distal ends curve downwardly in a near vertical orientation. When the electrode headset 330 is placed on a subject's head, the subject's head tends to urge the dorsal bands 356, 358 outwardly and upwardly, away from a center point of the electrode headset 330. The dorsal bands 356, 358 are formed from a material that includes enough flexibility to permit the subject's head to displace the bands 356, 358 without breakage, yet rigid enough that the dorsal bands 356, 358 conform to and embrace the subject's head and provide a snug fit thereto. The one or more electrodes included in the electrode apertures 346-349 and 331-334 are pressed against the subject's head with enough pressure to provide suitable contact at the electrode-subject interface to obtain a sufficient signal.

The center band 360 in this implementation includes three electrode apertures 335, 340 and 345, which can be used to mount one or more electrodes. In one implementation, as shown in FIG. 4, electrode aperture 335 is positioned to mount an electrode over the parietal lobe and electrode aperture 340 is positioned to mount an electrode over the occipital lobe. The electrode aperture 345 is positioned to mount an electrode over the parietal lobe.

In one implementation, the electrode aperture 335 can be eliminated and the electrode aperture 345 can be used to mount a reference electrode. In other implementations, a different electrode aperture can be used to mount a reference electrode (e.g., electrode aperture 335, in which case electrode aperture 345 can be eliminated). The particular position of the reference electrode in this implementation is illustrative.

In addition to providing electrode apertures 335, 340 and 345, the center band 360 provides a structure upon which the dorsal bands 356, 358 and temporal bands 352, 354 can be mounted and thereby properly positioned, such that electrode apertures included therein are properly positioned in accordance with a desired electrode placement scheme when the electrode headset 330 is worn by a subject.

A significant advantage of the substantially rigid design of the electrode headset 330 is that electrodes mounted therein are positioned in substantially predictable locations on the subject's head. Even though the electrode headset 330 includes some flexibility such that the various bands included in the headset 330 can conform to the subject's head and fit a variety of head shapes and sizes, the electrodes mounted therein will ultimately be located in substantially the same locations on each subject's head, i.e., in accordance with a desired electrode placement scheme. Because electrode placement is critical to obtaining the desired output signals from the electrodes, being able to provide reliable and accurate electrode placement is a significant advantage.

Referring again to FIG. 4, in the particular implementation shown, the electrodes when the electrode headset 330 is positioned on the subject's head, are spaced substantially according to the dimensions indicated on the drawing. The dimensions are shown in millimeters. For example, the distance between the electrodes mounted in electrode aperture 349 and electrode aperture 331 included in the left and right dorsal bands 356, 358 is approximately 85 millimeters. The other dimensions shown are approximate and are illustrative of the particular implementation shown. In other implementations, the bands can be configured differently and/or the electrode apertures can be positioned differently, so as to provide different spacing between electrodes mounted therein.

In another implementation, each of the left and right temporal bands 352, 354 can be lengthened by approximately 20 millimeters. With respect to the left temporal band 352, the distance between the electrode mounts 340 and 341 can be extended from 85 millimeters to 95 millimeters and the distance between the electrode mounts 341 and 342 can be extended from 51 millimeters to 61 millimeters. With respect to the right temporal band, the distance between the electrode mounts 340 and 336 can be extended from 85 millimeters to 95 millimeters and the distance between the electrode mounts 336 and 337 can be extended from 51 millimeters to 61 millimeters.

An advantage of the electrode headset 330 is that a single electrode can be removed and/or replaced independent of other electrodes mounted within the same electrode headset 330. This is an improvement over a conventional electrode headset that does not allow for individual electrode replacement, therefore rendering an entire headset unusable if one or more electrodes malfunctions or ceases operating. In the particular embodiment shown, some of the electrode apertures include a slot extending from the substantially circular opening to an outer edge. The slot can provide tension in the electrode aperture and facilitate insertion and removal of an electrode. In one implementation, an annular member is included within each electrode aperture and in one implementation is formed from acrylic.

In one implementation, the center band 360 can be used to either house or mount electronic circuitry that is electrically connected to the one or more electrodes mounted within the electrode headset 330. The electronic circuitry can be configured to receive signals from the electrodes and provide an output to a processor and/or may be configured to perform at least some processing of the signals. For example, referring again to FIG. 1, in some implementations electronic circuitry mounted on or housed within the electrode headset 330 can be configured to perform some or all of the functions of the sensor interface 104, A/D converter 106, data buffer 108, processing system 109 and/or platform 120.

In one implementation, the electrode headset 330 is substantially formed from a polystyrene material, although other materials can be used including nylon. Optionally, some regions of the electrode headset 330 can be reinforced with an additional layer or extra thickness of the same or a different material, for example, a polystyrene reinforcement layer. Optionally, pads can be included in some regions such that the pads make contact with the subject's head and resist slippage against the subject's head and/or to improve the fit and subject's comfort. In one implementation the pads are formed from silicon. Referring again to FIG. 3G, in the implementation shown, the reinforced regions include the regions 310a-f and the padded regions include the regions 312a-f.

Alternative Rigid Electrode Headset

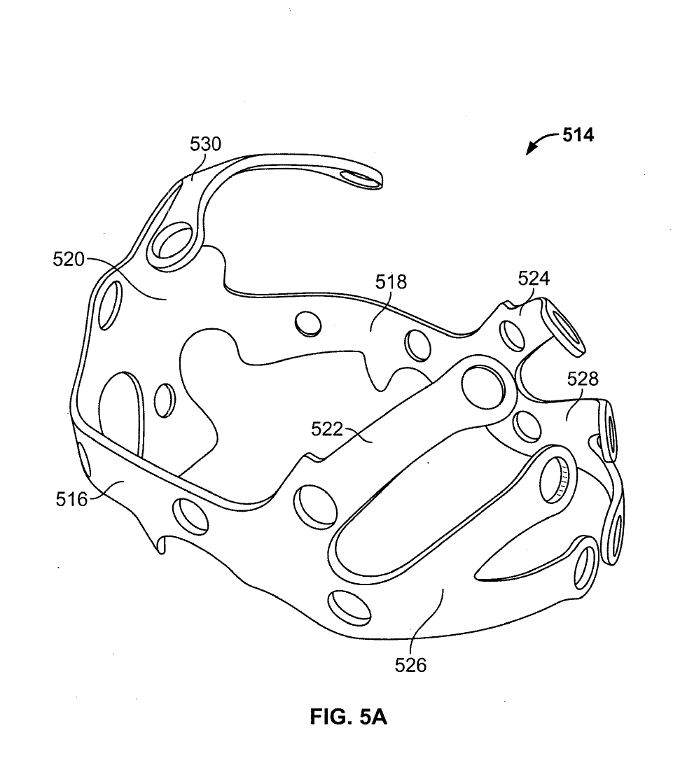

Referring now to FIGS. 5A-B, an alternative implementation of an electrode headset 514 is shown. The electrode headset 514 is formed from a rigid yet flexible material, and is configured to fit a range of head shapes and sizes, while maintaining suitable pressure of electrodes mounted therein against the subject's head. This particular implementation is configured to mount electrodes according to the same electrode placement scheme 350 shown in FIG. 4 as the electrode headset 330 described above. However, the orientation of the bands forming the electrode headset 514 within which the electrodes can be positioned according to the scheme 350 are different.

In this implementation, the electrode headset 514 includes two side bands 516, 518 extending from a center band 520. At distal ends of the two side bands 516, 518 are formed mid-bands 522, 524 and front bands 526, 528. Each front band is formed in a substantially V-shape and includes an upper portion and a lower portion. Additionally, an upper band 530 is connected to the center band 520 and extends over the back, top of the subject's head in a substantially V-shape. Each band includes one or more electrode mounts configured to mount an electrode therein, in a similar manner as described above in reference to the electrode headset 330.

In this implementation the electrode mounts are apertures configured to receive and mount an electrode therein. However, it should be noted that other configurations of electrode mounts can be used. For example, an electrode can be mounted to the electrode headset using a clamp, screw or other suitable connection mechanism and/or configuration.

Referring to FIG. 5B, an inner plan view of the electrode headset 514 is shown as if flattened out, to illustrate the electrode apertures 331-349. The electrode apertures are numbered with the same reference numerals as the electrode apertures shown in FIG. 3G for the electrode headset 330, since the correspondence to the electrode placement scheme 350 shown in FIG. 4 is the same.

In one implementation, the electrode headset 514 is substantially formed from a polystyrene material, although other materials can be used including nylon. The electrode headset 514 can optionally include reinforced regions to provide additional support. Optionally, the electrode headset 514 can include one or more padded interior regions, to resist slippage against the subject's head and/or to improve the fit and subject's comfort. The center band 520 can be configured to mount and/or house electronic circuitry that can be electrically connected to one or more electrodes mounted in the electrode apertures 331-349, similar to the electronic circuitry described above in reference to the electrode headset 330.

Another Alternative Rigid Electrode Headset

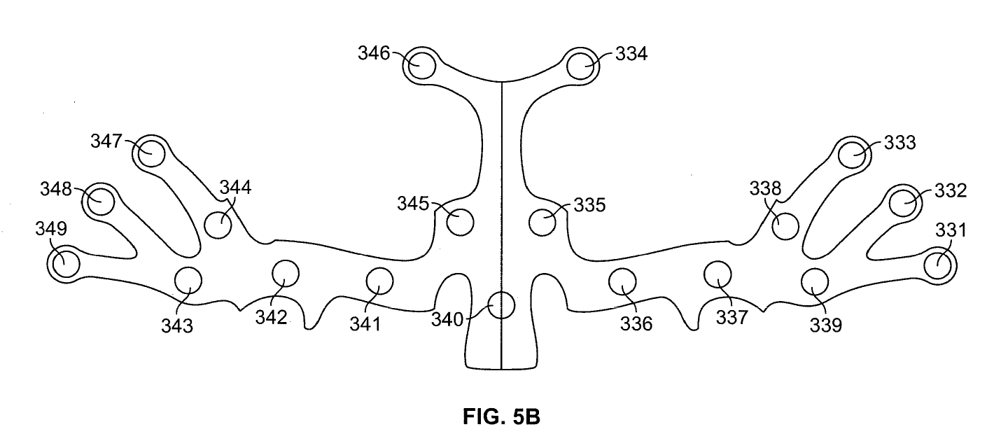

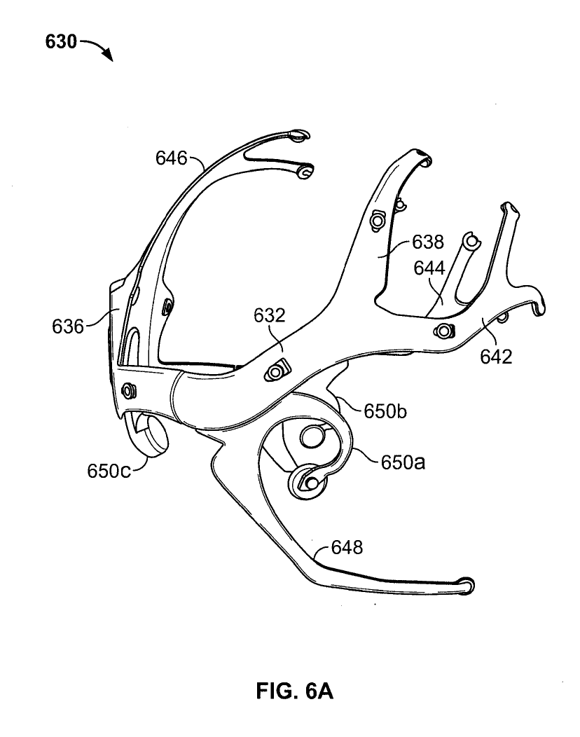

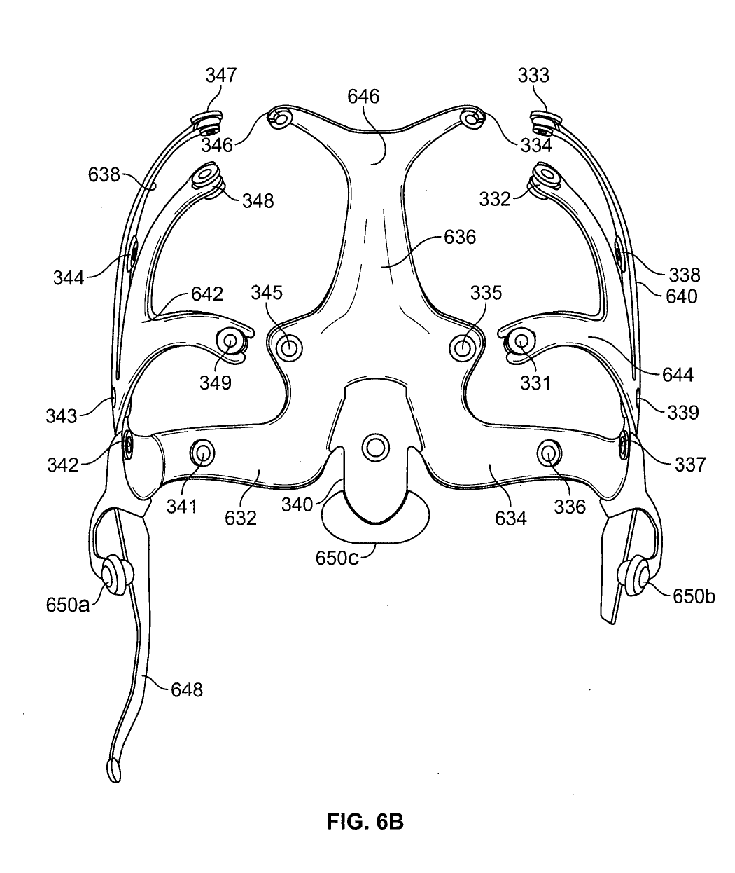

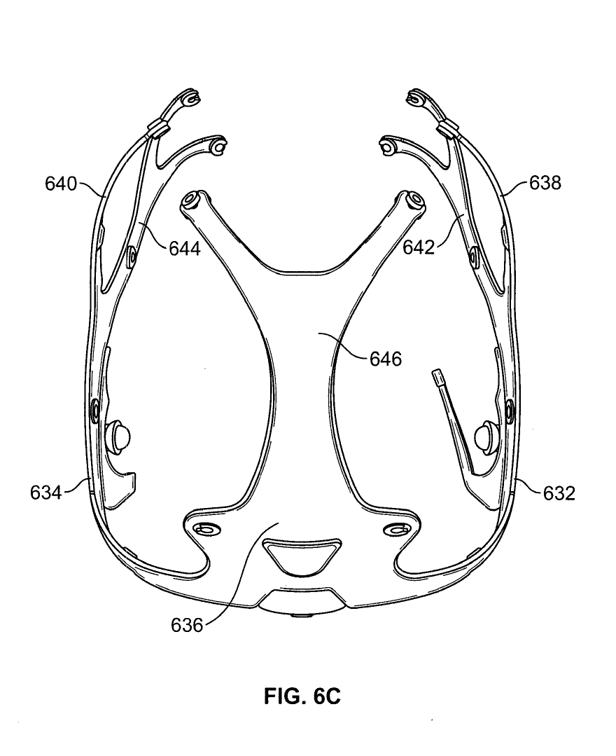

Referring to FIGS. 6A-C, another alternative implementation of an electrode headset 630 is shown. This electrode headset 630 has a similar configuration to the electrode headset 514 shown in FIGS. 5A-B and described above. The electrode headset 630 includes two side bands 632, 634, connected to a center band 636 on their proximal ends and to mid-bands 638, 640 and front bands 642, 644 on their distal ends. Additionally, an upper band 646 connects to the center band 636 and extends up and over a subject's head.

The electrode headset 630 includes electrode mounts positioned according to the same electrode placement scheme 350 shown in FIG. 4 and described above. In this implementation the electrode mounts are apertures configured to receive and mount an electrode therein. However, it should be noted that other configurations of electrode mounts can be used. For example, an electrode can be mounted to the electrode headset using a clamp, screw or other suitable connection mechanism and/or configuration.

For simplicity, the same reference numerals 331-349 are used to refer to the electrode apertures as are used in FIG. 4, to show the correspondence to the electrode placement scheme 350. In the depiction of this implementation, electrodes are shown mounted in the electrode apertures 331-349.

This implementation of the electrode headset 630 includes an optional chin strap 648 that can be used to snugly secure the electrode headset 630 to the subject's head. An optional chin strap can also be used in the other implementations of electrode headset 330 and 514 described above. Additionally, optionally pads mounted on extensions 650a-c are included to assist in positioning and comfort for the subject. In one implementation, the electrode headset 630 is substantially formed from a polystyrene material, although other materials can be used including nylon. Optionally, some regions of the electrode headset 630 can be reinforced with an additional layer or extra thickness of the same or a different material, for example, a polystyrene reinforcement layer.

In one implementation, the electronic circuitry is mounted on the electrode headset 630 and electrically connected to each electrode mounted therein by one or more wires extending between the electronic circuitry and each electrode. In another embodiment, the physical components electrically connecting the electrodes to the electronic circuitry, e.g., the wires, are embedded within the material forming the components of the electrode headset 630 and can be invisible and inaccessible to a user. This embodiment provides a sleeker, more compact design and functions to protect the wires extending between the electrodes and the electronic circuitry. For example, if the electrode headset 630 is formed from plastic components, wires connecting the electrodes to the electronic circuitry can be embedded within the plastic. Additionally, the electronic circuitry itself can be embedded within the plastic and made invisible to a user, for example, using a flexible printed circuit board (PCB).

Materials for Rigid Electrode Headsets

The electrode headsets 330, 514 and 630, can be formed from a material exhibiting one or more of the following qualities: highly durable and tough; providing a high degree of functionality; idea for designs including working snaps, fits and/or clips; good impact strength and capable of withstanding or resisting moisture and temperature.

In one implementation, the electrode headsets 330, 514 and 630 described above are substantially formed from a polystyrene material, although other materials can be used including nylon. Optionally, some regions of the electrode headset 630 can be reinforced with an additional layer or extra thickness of the same or a different material, for example, a polystyrene reinforcement layer. Optionally, pads can be included in some regions such that the pads make contact with the subject's head and resist slippage against the subject's head and/or to improve the fit and subject's comfort. In one implementation the pads are formed from silicon.

One example of such a material is SLS (Selective Laser Sintering) Cap Tuff General Purpose 25% Glass Filled Nylon 11 Material available from Envizage, a division of Concentric Asia Pacific, Melbourne, Australia. The SLS Cap Tuff material has a flexural modulus of 2020 Mpa, a tensile modulus of 2460 Mpa and a tensile strength of 38 Mpa. Other materials exhibiting one or more of the qualities described above can be used.

In another implementation, the WaterShed™ 11120 material available from DSM Somos of New Castle, Del., can be used. The WaterShed 11120 material is a durable, strong, semi-transparent, water-resistant resin. Other materials can be used and the ones described are examples.

Soft Electrode Headset

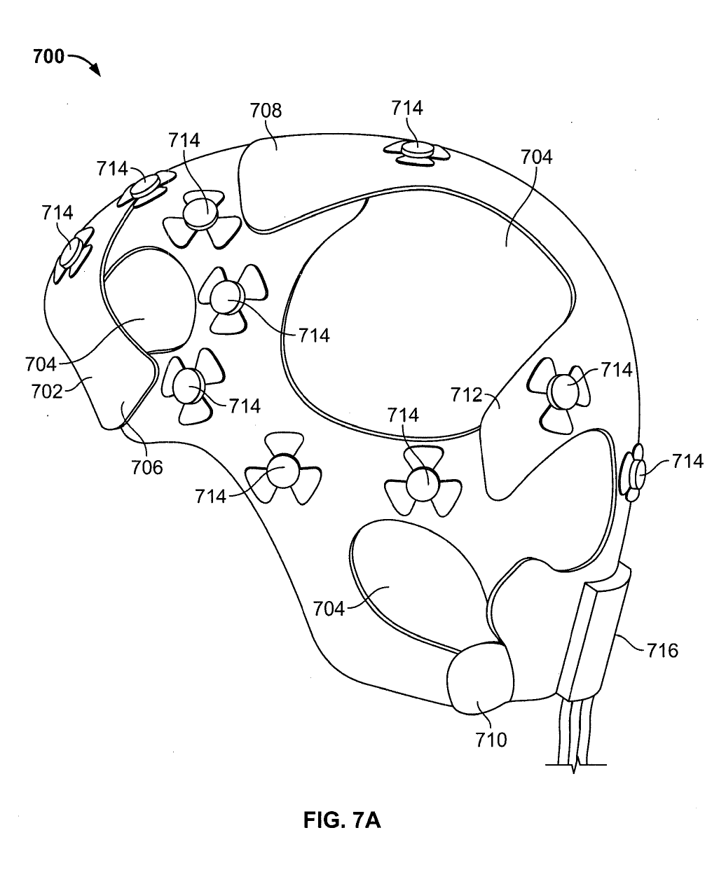

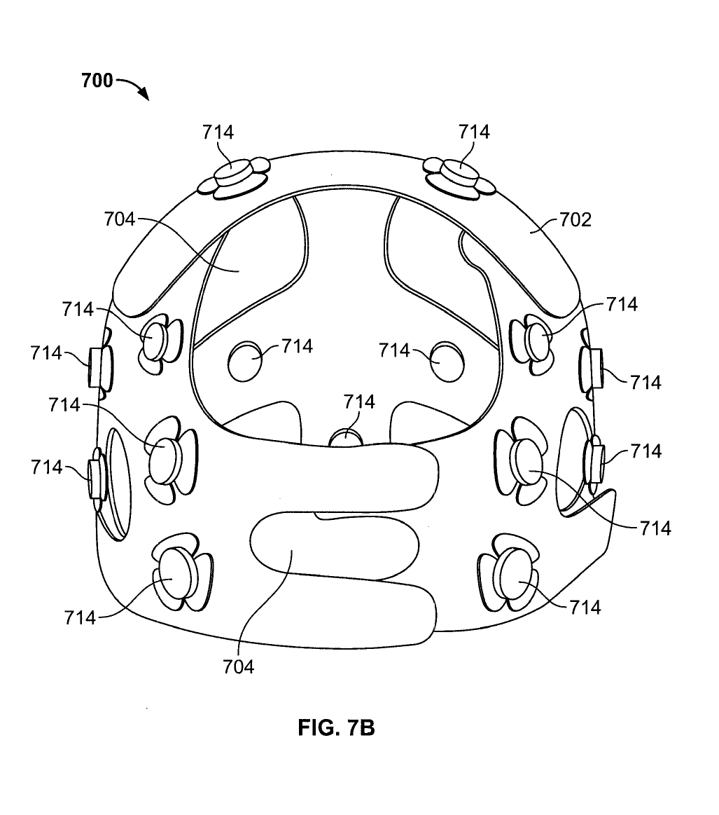

FIGS. 7A-B show an embodiment of a soft electrode headset. The electrode headset 700 is configured to position and hold in place one or more biosensors on a subject's head such that suitably accurate signals can be acquired from the subject. In one implementation, the biosensors are EEG electrodes, however, in other implementations different types of biosensors can be used.

In one implementation, the electrode headset 700 is shaped to fit to the contours of a subject's head without interfering with his or her vision, hearing or movement. The electrode headset includes a crown portion 702, which can be formed from a webbing, for example, made from a fabric material. The material used for the webbing can be a stretchable and soft material, for example, neoprene. A stretchable material can enable the electrode headset 700 to be worn securely while still being comfortable to the subject. The webbing of the crown portion 702 includes voids 704 to allow airflow to the subject's head to prevent overheating and improve comfort. A number of adjustable portions are provided, for example, components 706, 708, 710 and 712, which can be formed from overlapping webbing sections joined together with connectors that can be used to adjust the size of the headset. In one implementation, the connectors are hook and loop fasteners (e.g., Velcro®), although other forms of connectors can be used.

In various locations on the soft electrode headset 700 are included electrode mounts configured to mount an electrode, such as electrodes 714. In the implementation shown, the electrodes 714 are included within electrode mounts configured as apertures formed through the crown webbing material. The webbing material is sufficiently stretchy and resilient and the apertures are sized such that an electrode mounted therein is securing held in place. In one implementation, the apertures have a substantially triangular shape.

Each electrode is connected by one or more wires to electronic circuitry 716, which is described further below. The wires can be concealed in channels formed within the electrode headset 700 or held by loops of material formed into, or attached along, the crown webbing. The channels or loops can be formed on the inside or outside of the electrode headset 700.

The wires extend between each electrode and the electronic circuitry 716. In one implementation, the electronic circuitry includes an SCSI connector, although other connectors that can accommodate the necessary number of wires and that are sufficiently lightweight can be used. If the connector is too heavy, the connector may annoy the subject, impede his or her head movement or cause the electrode headset 700 to move on the subject's head.

In one implementation, the electrode headset 700 includes 19 electrode mounts to mount therein 17 electrodes for taking EEG measurements, one ground electrode and one reference electrode. Referring again to the electrode placement scheme 122 shown in FIG. 2, the 17 electrodes can occupy the following electrode positions included in the “10-20” scheme 122: FP1, FP2, AF3, AF4, F3, F4, F7, F8, FC5, FC6, T7, T8, P7, P8, PO3, PO4 and OZ. The ground electrode and reference electrode can occupy positions CP1 and CP2.

Electrodes mounted within the electrode headset 700 can be expensive and an advantage of the electrode headset 700 is that the number of electrodes mounted therein can be increased or decreased by the subject to suit his or her needs. For example, in a certain application, e.g., as detecting an emotion, the electrode headset 700 may only need a small number of electrodes mounted therein, while for another application, e.g., detecting a conscious effect such as to move a real or virtual object or a muscle movement, one or more additional electrodes may be needed.

Referring to FIG. 8, one implementation of an alternative electrode mount configuration is shown. In this implementation, the electrode mount is an electrode pocket 820, and a cutaway view is shown in the figure. The electrode pocket is formed within the material 822 forming the electrode headset 700 and an electrode 814 is mounted therein. The electrode pocket 820 is generally square in shape and includes an access opening 836 through which the electrode 814 can be inserted. The material 822 is stretchable and resilient and thus the access opening 836 can be sized smaller than the electrode 814. The electrode 814 is received into the electrode pocket 822 and mounted such that a contact portion of the electrode 814 extends through an aperture 830 formed in the bottom face 838 of the electrode pocket so as to contact the subject's head.

Electrode

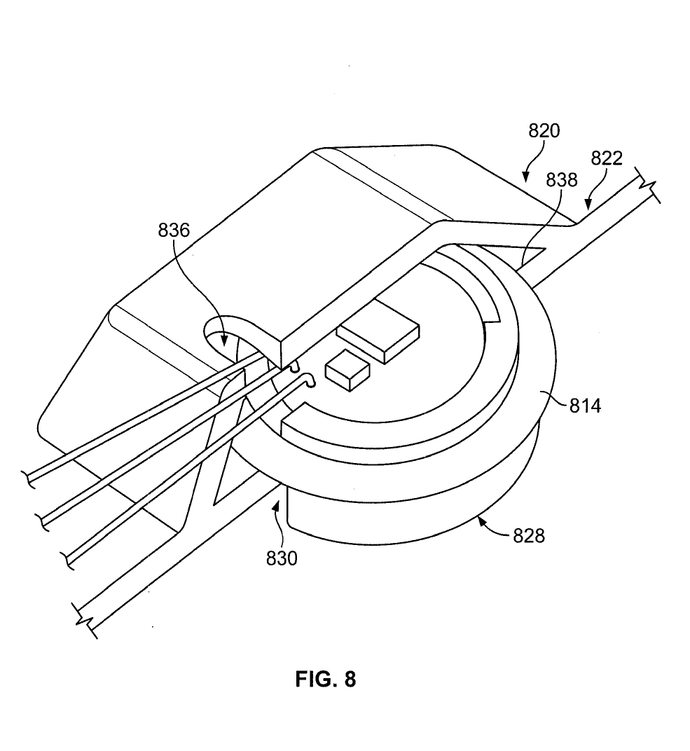

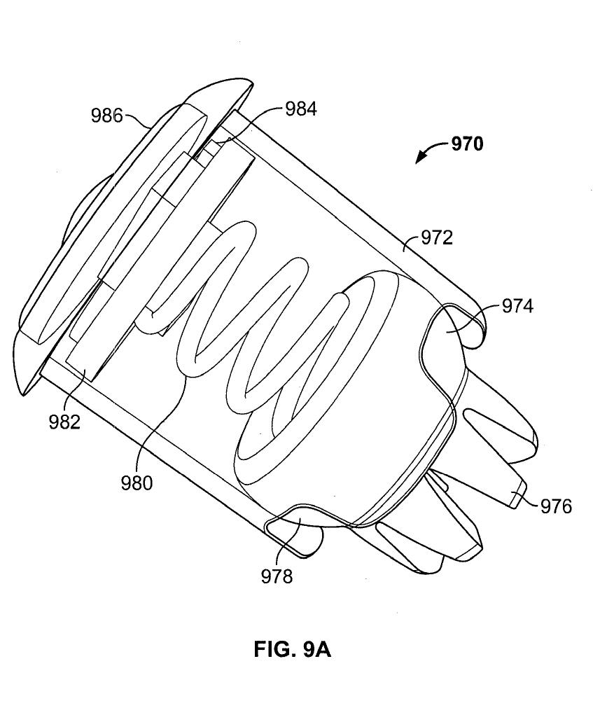

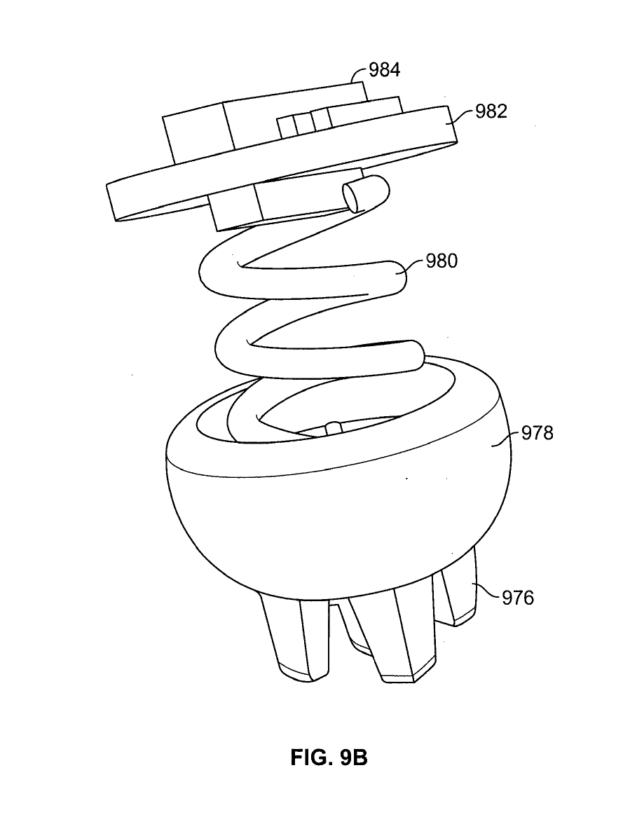

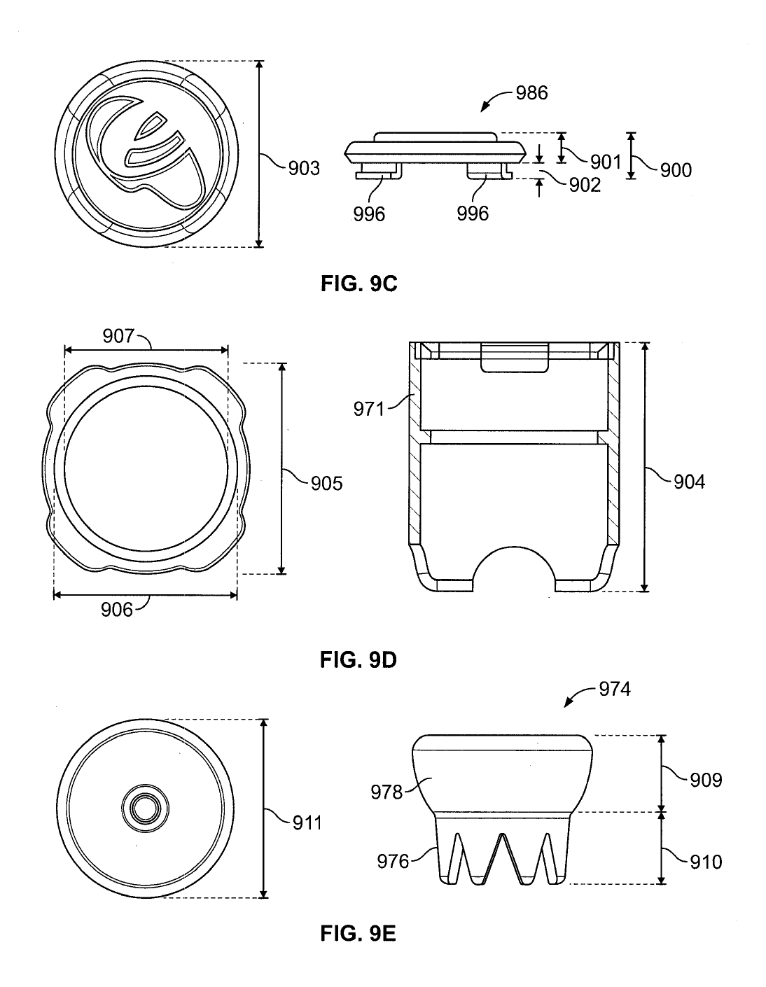

Referring to FIGS. 9A-F, one implementation of an electrode 970 that can be mounted within the electrode headset 330, or used independent of the electrode headset 330 for a different application, is shown. In this implementation, the electrode 970 is configured as an active resistive electrode. The electrode includes a housing 972, which for illustrative purposes is shown as transparent, including a substantially tubular body 972 and a cap 986. Referring particularly to FIG. 9B, the electrode 970 is shown with the housing 972 removed for illustrative purposes. The electrode 970 includes a printed circuit board (PCB) 984 attached to an electrode plate 982. The PCB 984 includes electronic circuit components forming a sensor circuit. One or more wires can connect to the sensor circuit to provide power to the circuit and permit signals to be sent from the sensor circuit to a signal acquisition system, which can be mounted or housed within the electrode headset 330 or located external to the electrode headset 330.

A flexure element 980 is attached to the underside of the electrode plate 982 and connects on a second end to a gimbaled contact 974. In this implementation the flexure element 980 is a spring, although in other implementations the flexure element can be configured differently. The gimbaled contact 974 includes an upper portion 978 forming a gimbaled connection to the housing 972. A lower portion of the gimbaled contact provides one or more contact elements 976 configured to contact the subject's skin. The flexure element 980 is formed from a conductive material, thereby electrically connecting the gimbaled contact 974 to the electrode plate 982. A conductive path is thereby provided from the subject's skin to the electrode plate 982 via the gimbaled contact 974 and flexure element 980. Bioelectrical potentials from the subject's skin detected by the gimbaled contact 974 are thereby provided to the electrode plate 982 and ultimately to the sensor circuit included in the PCB 984.

The flexure element 980 can be made from a conductive material, for example, a metal. The electrode plate 982 can be made from biocompatible metal or biocompatible metal alloy and in one implementation is formed from silver-silver-chloride (AgAgCl). The electrode plate 982 material selection is important to ensure proper biosignal acquisition and minimize skin-electrode noise. Other example materials include: silver, gold and tin, but are not limited to these.

In one implementation, the electrode 970 can function as a dry electrode 970, meaning a sufficient signal can be received at the gimbaled contact 974 and transmitted to the sensor circuit without using a wet, conductive material, i.e., a conductive gel, fluid or wetted contact pad, at the electrode-skin interface; the contact elements 976 can make direct contact with the subject's skin. In another implementation, to improve signal strength, the electrode 970 can function as a wet electrode. That is, the electrode 970 can be used in conjunction with a wet conductive material, such as a conductive gel or fluid or a wetted contact pad. In one particular implementation, a contact pad formed from a material suitable to retain a conductive fluid, e.g., a felt pad, and wetted with the conductive fluid can be placed between the contact elements 976 and the subject's skin.

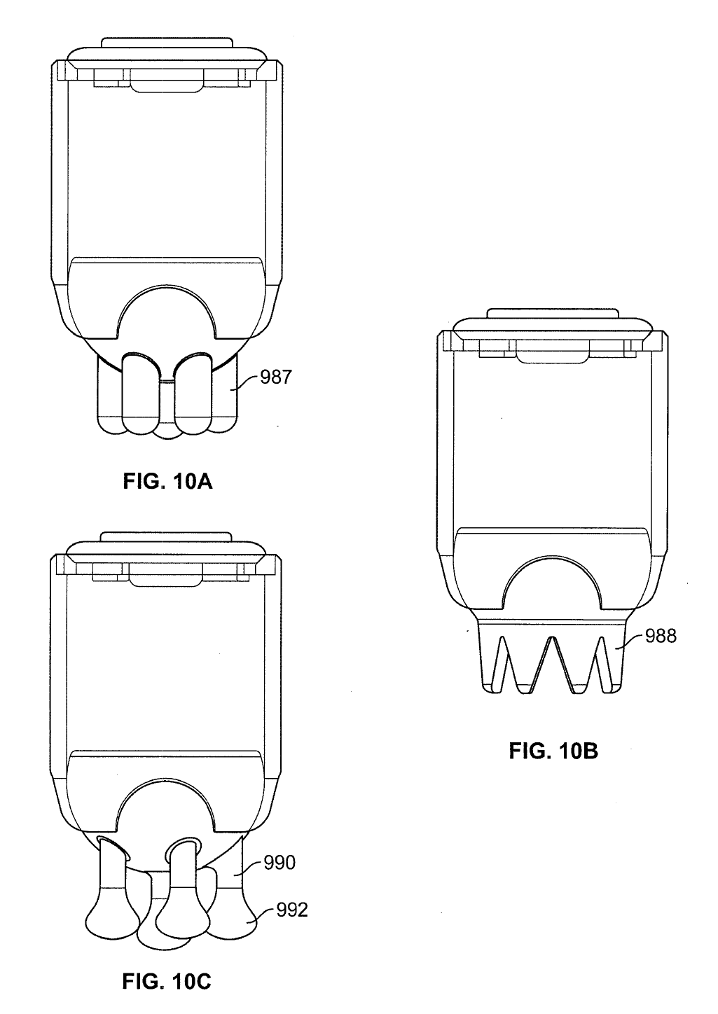

Various embodiments of the contact elements 976 can be used. In an implementation where the electrodes will be used on a subject's head, preferably the contact elements 976 are formed as elongated protrusions as shown, to provide sufficient contact with the subject's skin through the subject's hair. Referring to FIGS. 10A-C, alternative implementations of the contact elements are shown. In FIG. 10A, the contact elements 987 are substantially cylindrical with rounded ends. In FIG. 10B, the contact elements 988 are substantially triangular shaped. In FIG. 10C, the contact elements 990 are substantially cylindrical and include bulbous tips 992.

Referring now to FIGS. 9D-F, the housing 970 and gimbaled contact 974 are shown in further detail. The housing includes a substantially tubular body 971 and a cap 986. In the particular implementation shown, the cap 986 includes projections 996 configured to provide a snap fit connection to the tubular body 971, by snapping underneath a rim provided at an upper surface of the tubular body 971. The tubular body 971 includes an interior region configured to receive and house the upper portion 978 of the gimbaled contact 974. The gimbaled contact 974 includes rounded, conical shaped sides, which fit within the lower portion of the interior region of the tubular body 971 and are configured to permit the gimbaled contact 974 to tilt freely in all directions within the housing 972.

Preferably, to receive a suitable signal, the contact elements 976 are positioned substantially perpendicular to the subject's skin when the electrode headset 330 is worn by the subject. An advantage to the gimbaled contact 974, is that some relative movement between the electrode headset 330 and the subject's head can occur, while maintaining some contact between the contact elements 976 and the subject's skin in the preferred orientation. The flexure element 980 allows the distance from the electrode plate 982 and the contact elements 976 to vary within a certain range determined by the amount of flex permitted by the flexure element 980. Further the gimbaled contact 974 can gimbal, i.e., swivel and/or tilt, within the housing 972. As such, with the distance between the electrode plate 982 and the contact elements 976 permitted to vary, and the gimbaled contact 974 able to tilt, even if the housing 972 changes position such that the tubular body 971 is not substantially perpendicular to the subject's skin, the gimbaled contact 974 can reorientate within the tubular body 971, such that the contact elements 974 maintain a position substantially perpendicular to the subject's skin. Accordingly, the preferred orientation can be maintained and a suitable signal received, even with some shifting of the electrode headset. Given that in some applications, particularly in a non-clinical setting, some movement of the subject's head is almost always occurring, the gimbaled contact gives the subject a more enjoyable and hands off experience, as the electrode headset does not require constant adjustment.

In one implementation, the housing 972 is formed from plastic. The gimbaled contact including the contact elements can be formed from a biocompatible conductive material, for example, metal.

Referring now to FIGS. 3A and 9A, in one implementation, the tubular body 971 of the electrode is configured to friction fit within an electrode aperture included in the electrode headset 330. As described above, the electrode apertures can include an annular member that facilitates a friction fit to the outer surface of the tubular body 971. As previously described, each electrode 970 can be independently mounted within and removed from the electrode headset 330, allowing different subsets of electrodes to be used and allowing malfunctioning or broken electrodes 970 to be replaced.

Referring again to FIGS. 9C-E, the dimensions for one particular implementation of the electrode 970 shall be described. It should be understood however that other dimensions and relative dimensions can be used, and the ones described herein are illustrative of one embodiment. The cap 986 can have an overall height 900 of approximately 2.6 millimeters, including an upper thickness 901 of 1.6 millimeters and an approximate height 902 of the projections of 1 millimeter. The outer diameter 903 of the cap 986 can be approximately 11.2 millimeters. The tubular body 971 can have an overall height 904 of approximately 15 millimeters. The overall outer diameter 905 can be approximately 12.7 millimeters, the inner diameter 906 can be approximately 11.2 millimeters and the inner ring diameter 907 can be approximately 10 millimeters. The gimbaled contact 974 can have an overall height 208 of approximately 8.8 millimeters including an approximate upper portion height 909 of 4.6 millimeters and an approximate contact element height 310 of 4.2 millimeters. The approximate outer diameter 911 of the top of the upper portion can be 10.8 millimeters.

An electrode headset 330 configured to receive an electrode 970 having the dimensions described above can include electrode apertures having an inner diameter sized to friction fit the tubular body 971 of the electrode 970. Accordingly, for an electrode 970 having a tubular body 971 with an outer diameter of approximately 12.7 millimeters, the inner diameter of the electrode aperture is also approximately 12.7 millimeters. As described above, these dimensions are examples of one embodiment. The inner diameter of the electrode apertures can vary, depending on the electrode to be mounted therein. In one implementation, the electrode apertures can have different inner diameters relative to one another, for example, if different sizes or types of electrodes are intended to be mounted in the various different electrode apertures.

Alternative Electrode

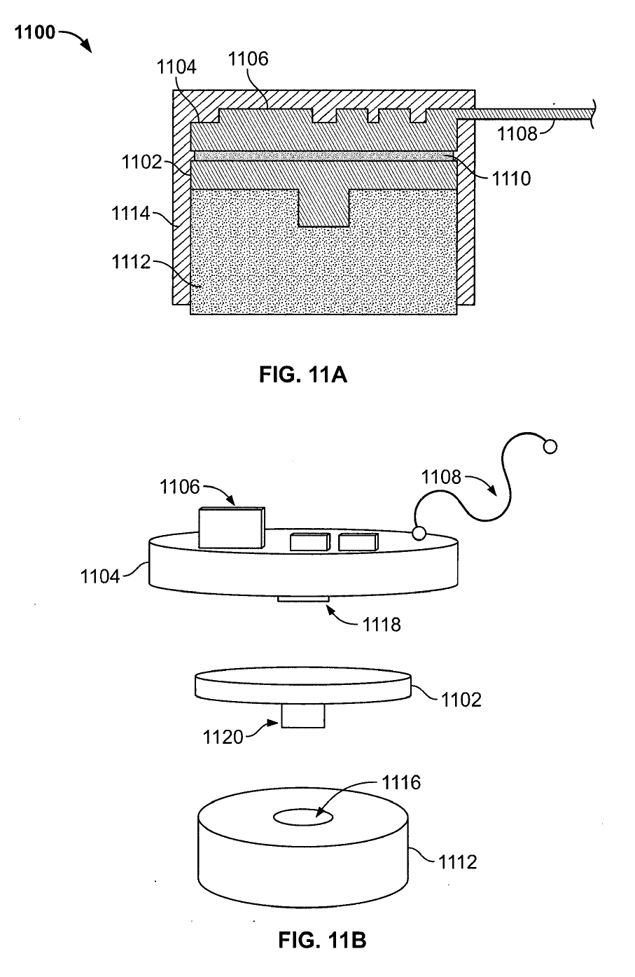

In addition to the electrode 970 described above, other configurations of wet or dry electrodes can be mounted within the electrode headsets described herein. Referring to FIG. 11A, a schematic cross sectional view of another implementation of an electrode that can be used in the electrode headsets described herein, or in another type of mounting structure for the same or a different application, is shown. The electrode assembly 1100 includes an electrode plate 1102 mounted to a printed circuit board (PCB) 1104. The PCB 1104 includes electronic circuit components forming a sensor circuit (denoted generally as 1106). One or more wires 1108 are connected to the sensor circuit 1106 to provide power to the circuit 1106 and permit signals to be sent to a signal acquisition system. The circuit 1106 of the PCB 1104 includes at least one electrical contact (not shown) that is configured to be connected to an electrode.

The electrode can be used to pick up bioelectrical potentials from the skin of a subject, and includes the electrode plate 1102. The electrode plate 1102 is maintained in electrical contact with at least one contact mounted on the PCB via a conductive medium, for example, a conductive glue 1110. On the underside of the electrode plate 1102 is mounted a contact pad 1112, which is configured to provide a conductive path between the subject's skin and the electrode plate 1102 when in use. Preferably the contact pad can hold a conductive liquid, such as saline solution, to improve electrical conductivity. However, in some implementations the electrode assembly can be used without a conductive liquid. The sub-assembly including the PCB 1104 and electrode plate 1102 can be waterproofed and mounted with the contact pad 1112 within a housing 1114.

FIG. 11B illustrates a schematic exploded view of the PCB 1104, electrode plate 1102 and contact pad 1112 shown in FIG. 11A. A circuit 1106 as depicted in FIG. 13 is formed on the PCB 1104. On the underside (or other convenient location) of the PCB 1104 is a conductive contact 1118. The conductive contact 1118 can be made of copper or another suitably conductive material, and is used to make electrical contact between the sensor circuit 1106 mounted on the PCB 1104 and the electrode plate 1102. One embodiment of the electrode plate 1102 is made of silver-silver chloride (AgAgCl) and is generally disk-like in shape. An upper surface of the electrode plate 1102 is maintained in electrical contact with the contact 1118, either directly or via a conductive material such as a silver epoxy conductive glue. The bottom surface of the electrode plate 1102 makes contact with the contact pad 1112, which can be made from a felt material, or include a felt material layer or portion.

On the underside of the electrode plate 1102 is a generally cylindrical projection 1120. The projection 1120 is configured to be received into a correspondingly shaped recess 1116 formed in the upper side of the contact pad 1112. The protrusion 1120 is sized to as to be a friction fit with the receiving hole 1116 in the contact pad 1112, and to thereby provide a secure mounting arrangement for fixing the contact pad 1112. The projection 1120 also increases the amount of surface area of the electrode plate 1102 that makes contact with the contact pad 1112, and therefore can increase the quality of signal acquisition. However, in alternative embodiments the mating surfaces of the electrode plate 1102 and contact pad 1112 can be flat, or can have an alternative shape or can be attached together differently.

In use the contact pad 1112 can absorb and hold electrolytic solution such as saline solution or other electrically conductive liquid and maintain a flexible and high quality conductive link between the subject's skin and the electrode plate 1102. The use of conductive liquid assists this process, but may not be essential in some embodiments. The contact pad 1112 can be made of an absorbent material, such as a felt sponge. For example, the felt sponge used in a dry printset self inking stamp, or felt used in a poster pen or similar “felt-tipped” pen, have suitable absorption and hardness properties for use in embodiments of the present invention, although other materials can be used. In order to protect the electronics of the electrode assembly from damage and to improve the safety of the electrode, the PCB can be enclosed in a waterproof housing. The waterproofed PCB and the attached electrode plate arrangement is inserted into the housing 1114.

In some embodiments, such as the embodiment shown in FIGS. 11A and 11B or the embodiment shown in FIGS. 9A-E, the electrode casing includes a plastic component of unitary construction. The casing can be tubular in configuration and serve a dual role of ensuring mechanical strength of the electrode arrangement and have an open end that can serve as a feed tube, through which electrolyte solution can be introduced to the contact pad 1112. The inside of the recess into which the PCB-electrode arrangement is received can include one or more retaining formations configured to hold the PCB-electrode arrangement and contact pad in place during use. The assembly can include a closure or other means to secure the PCB-electrode arrangement in the housing. Moreover, in one embodiment the housing 1114 can be configured to hold the PCB-electrode arrangement in a releasable manner to facilitate replacement of the PCB-electrode arrangement within the housing. The inside of the housing 1114 can be provided with teeth or circumferential ribs to hold the PCB-electrode arrangement in place, and allow the PCB-electrode arrangement to be pushed out for replacement. The replacement process requires connecting the replacement PCB-electrode arrangement into the acquisition system. In one implementation, this can be achieved using a known crimping or modular wiring/connector systems.

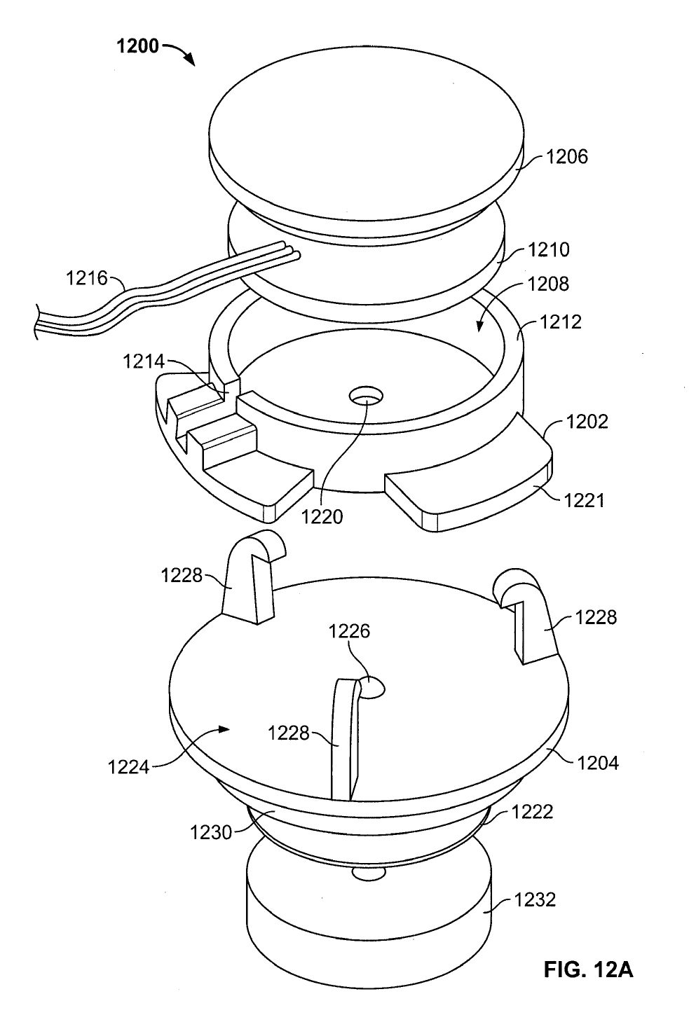

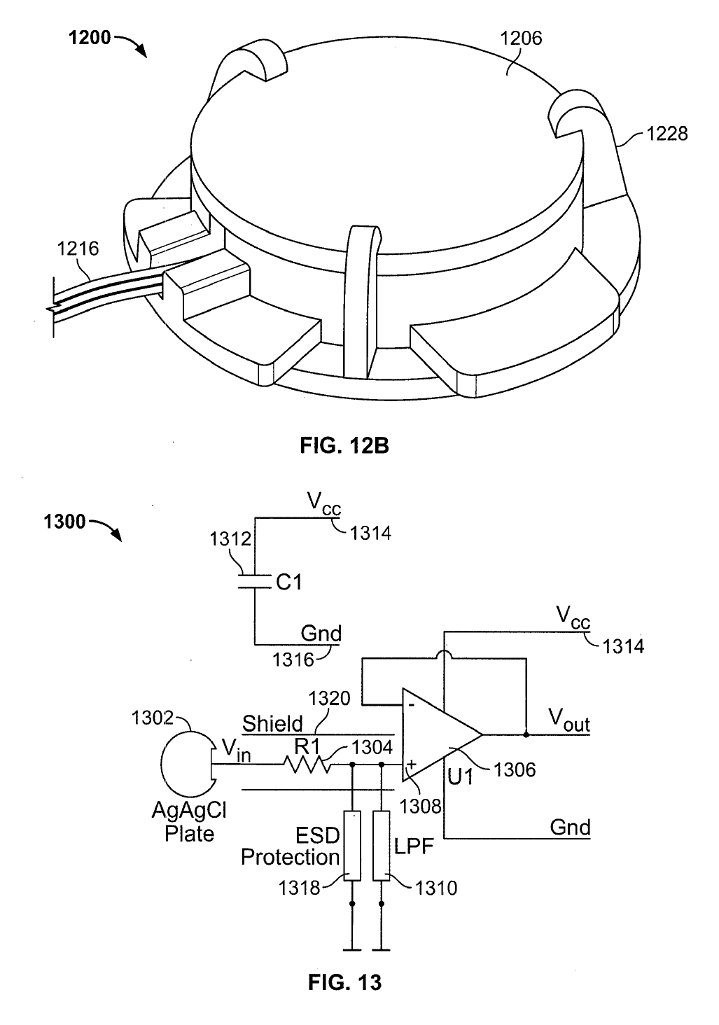

Referring to FIGS. 12A-B, an alternative electrode assembly is shown that can be used in an electrode headset described herein, or in a different mounting structure for the same or a different application. The electrode assembly 1200 of this embodiment includes a PCB receiving portion 1202, a base portion 1204 and a cap 1206. The PCB receiving portion 1202 includes a cavity 1208 and is preferably waterproofed, using a material that can also be used to hold the PCB 1210 in place in the housing. An opening 1214 allows wires 1216 to extend to the PCB 1210. The floor 1218 of the cavity 1208 is provided with an aperture 1220 to enable an electrical connection to be made between an electrode circuit on the PCB 1210 and an electrode plate 1222. The PCB receiving portion 1202 also includes one or more radial projections 1221, described further below.

A cap 1206 is provided that is configured so as to close off the cavity 1208 and hold the PCB 1210 in place within the housing. The base 1204 is mounted below the PCB receiving portion 1202, and includes a base portion 1224 with a through hole 1226. The through hole 1226 is provided to enable an electrical connection to be made, through the base 1204, between a contact of the electrode circuit on the PCB 1210 and an electrode plate 1222.

The base 1204 also includes a plurality (three in this embodiment) of retaining members 1228 that, when the housing is assembled, clip over the edge of the cap 1206 and retain the cap 1206 in place. The underside of the base 1204 further includes an annular flange 1230, that defines a recess into which the electrode plate 1222 is mounted. The electrode plate 1222 can be attached to the bottom of the base 1204 using, for example, a conductive glue. In use, sufficient glue is used to mount the electrode plate 1222 to the base 1204 such that the voids formed by the through holes 1226 and 1220 are substantially filled and electrical contact is made with a contact of the electrode circuit on the PCB 1210. A contact pad 1232 is mounted on the electrode as described in connection with the previous embodiment.

FIG. 12B depicts the electrode assembly of FIG. 12A in an assembled state. The electrode housing components can be made from a plastic material such as polyurethane. Such components can be made using from RTV molds created from fabricated styrene masters. Moreover in these embodiments the housings can have one or more electrolyte feed ducts that bypass non-waterproofed electronic components (or be configured to receive an external tube) that can enable electrolyte fluid to be applied to the contact pad of the electrode assembly in use. Such ducts can preferably allow application of the electrolyte fluid without removal of the electrodes from the subject.

It should be noted that since, electrode assemblies can be expensive it is advantageous to enable the number or electrodes to be increased and decreased by the subject to suit his or her needs. For example, an electrode headset in a certain application, e.g., detecting an emotion, may only need eight electrodes, whilst for another application, e.g., additionally detecting a conscious effort such as to move a real or virtual object, or a muscle movement, one or more additional electrodes may be needed. Therefore the electrodes should be mountable and detachable from the headset, e.g., electrode headsets 330, 514 and 530, for example, in the manner discussed above.

Circuit Diagram

Referring now to FIG. 13, a schematic circuit diagram is shown for an embodiment of an active electrode for sensing bioelectric potentials. The circuit 1300 depicted is suitable for use with an electrode or electrode assembly such as those shown in FIGS. 9A-E, 11A-B and 12A-B. The circuit 1300 includes an electrode plate 1302, that is maintained in electrical contact (directly or via a conductive path) with the subject's skin. For example, the electrode plate 1302 can be the electrode plate 982 of the electrode 970 shown in FIGS. 9A-E, the electrode plate 1102 of the electrode assembly 1100 shown in FIGS. 11A-B, or the electrode plate 1222 of the electrode assembly 1200 shown in FIGS. 12A-B.

The electrode plate 1302 provides an input voltage (Vin) that is initially applied to an input protection resistor R1 1304. The input resistor R1 1304 serves as overcurrent protection in case of electrode malfunction, and protects both the operational amplifier U1 1306 and the subject. In one embodiment, R1 1304 is a 5 kΩ resistor. The input resistor R1 1304 is connected to a positive terminal 1308 of the operational amplifier U1 1306. The operational amplifier U1 1306 can be set up in a buffer amplifier arrangement. In this example, the buffer amplifier has a gain of 1, however other gains can be used. The operational amplifier U1 1306 can be a CMOS operational amplifier, which provides a large input impedance, e.g., in the gigaohm range. The operational amplifier U1 1306 can have a lower output impedance than a passive electrode, and reduce hum caused by environmental interference, such as power line noise. The operational amplifier U1 1306 can have low intrinsic noise in the frequency range of 0.1 to 40 Hz, in order to enable accurate detection of weak EEG signals such as evoked potentials. The operational amplifier U1 1306 preferably has low drift and low offset voltage.

In one embodiment, the operational amplifier U1 1306 is a Texas Instruments operational amplifier model No. TLC2201. Alternatively a TLV2211 operational amplifier (also from Texas Instruments) can be used and may be advantageous, as it has a smaller footprint and lower current consumption. As will be known to those skilled in the art other types of operational amplifiers can be used, e.g., a OPA333 operational amplifier (also from Texas Instruments). The circuit 1300 includes an optional low pass filter (LPF) 1310, which can be used to filter out noise introduced by sources such as radio frequency interference and that can affect the quality of signals required by the electrodes. The circuit 1300 also includes optional electro static discharge (ESD) 1318 protection circuitry to protect the operational amplifier U1 1306 in case of electrostatic discharge. The circuit 1300 includes a bypass capacitor C1 1312 connected between the power supply signal Vcc 1314 of operational amplifier U1 1306 and to ground 1316 to decouple the power supply. An optional PCB shield 1320 can be included around an input trace.

Electrode Assembly Housing

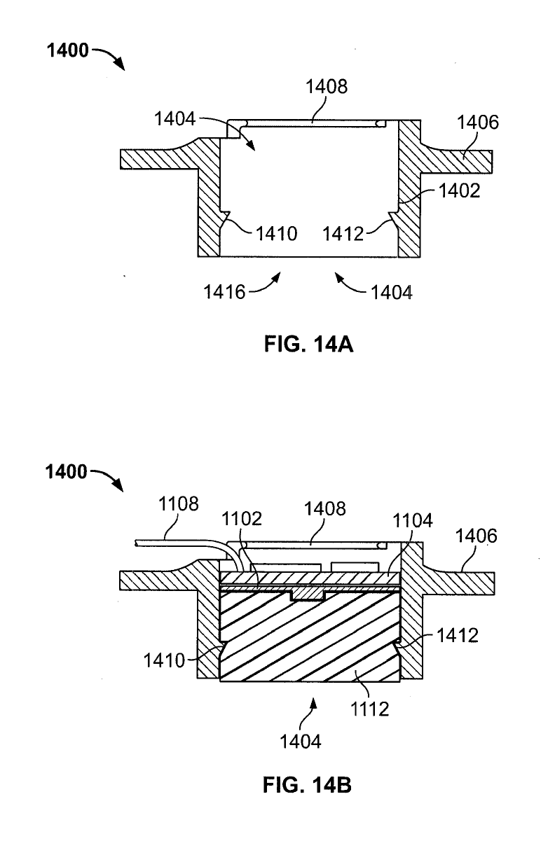

Referring now to FIG. 14A, a cross-sectional view of one implementation of an electrode assembly housing is shown. As can be seen the main body 1402 of the housing 1400 is generally cylindrical and defines a chamber 1404 into which the PCB, components of the acquisition circuit, an electrode plate and contact pad can be mounted in use. The housing 1400 further includes a radially extending flange 1406 to prevent the electrode assembly from being pushed through an electrode aperture of an electrode headset within which the housing 1400 is mounted. At the top of the chamber 1404 is located a plurality of inwardly extending flanges 1408 to prevent the components installed in the chamber 1404 from being pushed out of the top of the chamber 1404. The inner wall defining the chamber 1404 can include at least one tooth 1410, 1412 or rib(s) to retain the contact pad in the chamber 1404.

To assemble the electrode assembly, a pre-assembled PCB-electrode plate arrangement can be slid under the flanges 1408 in the direction of arrow 1414 until located in the chamber 1404. The contact pad can be inserted into the bottom of the chamber 1404 in the direction of arrow 1416 and installed in contact with the PCB-electrode plate assembly.

FIG. 14B shows the electrode assembly housing depicted in FIG. 14A with the electrode components assembled therein. In this implementation, the electrode assembly included in the housing 1400 is the embodiment shown in FIGS. 11A-B. Referring to the same reference numerals of FIGS. 11A-B, the printed circuit board (PCB) 1104 is mounted in the topmost position in the chamber 1404, followed by the electrode plate 1102. The contact pad 1112 is bottom-most in the chamber 1404, and is located in contact with the electrode plate 1102. The contact pad 1112 is secured in the chamber 1404 by teeth 1410 and 1412, which grip the sides of the contact pad 1112. Using such an arrangement, the components can be easily removed from the housing and replaced if a malfunction occurs in the electrode's components.

Signal Acquisition System

As described above, each electrode headset is configured to mount therein one or more electrodes. Each electrode is electrically connected to electronic circuitry that can be configured to receive signals from the electrodes and provide an output to a processor. The electronic circuitry also may be configured to perform at least some processing of the signals received from the electrodes. In some implementations electronic circuitry mounted on or housed within the electrode headset can be configured to perform some or all of the functions of the sensor interface 104, A/D converter 106, data buffer 108, processing system 109 and/or platform 120.

In one implementation, the electronic circuitry is mounted on the electrode headset and electrically connected to each electrode mounted therein by one or more wires extending between the electronic circuitry and each electrode. The wires can be either visible on the exterior or interior of the electrode headset, or can be formed within the electrode headset, for example, by molding within one or more plastic components forming the electrode headset. Preferably, the wiring system exhibits one or more of the following features: a low cost; termination at the electronic module with a connector; flexible and shapeable to fit the contour of the electrode headset; strain relief at the conductor terminations; non-breakable flexible wiring with strain relief, moldable in a rigid headset; noise immunity and having conductor resistance less than 100 ohms.

In one implementation having eighteen electrodes mounted within an electrode headset includes two flex cables accommodating a total of 52 wires. In this particular implementation 52 wires are grouped into 2 flexible cables of 28 and 24 pins respectively. Both flexible cables are 1 mm in pitch and are laminated to add to durability and prevent from damage. The wires are adjoined to electrode circuitry through soldering or crimping means, but adjoinment is not limited to these methods. In one implementation wires can be cut to length, crimped and inserted into the connector housing, twisted and stripped by machinery.

In another implementation having eighteen electrodes mounted within an electrode headset, the wiring system includes two connectors accommodating a total of 52 wires. Each wire is multi-stranded and PVC insulated. For example, each multi-stranded wire can include seven 0.26 millimeter diameter conductors. The wires are crimped with receptable contacts and inserted into the connector housing. The wires leading to each electrode board are grouped and twisted together and then stripped at the ends and soldered to the electrode PCB. In one implementation, the wire can be cut to length, crimped, inserted into the connector housing, twisted and stripped by machinery.

In the above implementation, one or more of the following advantages can be realized. The material cost can be minimized (low cost connectors and minimal or no wire wastage, as each wire is cut to the exact length). The wire being flexible can fit the contour of the electrode headset. The manufacturing process is efficient as it can be automated. Twisting the wires can serve to provide noise immunity and efficient placement of the wiring into the headset, as all three wires twisted together is similar to a single mini-cable. The cost of terminating the wire to the electrode board can be minimal, as it can be soldered to the electrode PCB. Strain-relief requirements can be less critical because the wire is multi-stranded, although inexpensive effective strain relief can be provided by threading the wire through another hole in the PCB or alternatively by including a strain relief in the electrode headset molding itself. The wiring assembly can tolerate temperatures of common plastic injection molding processes (e.g., polyethylene at 115 degrees Celsius), as the connectors can be made from nylon and the wire can be PVC insulated and therefore be expected to withstand approximately 150 degrees Celsius. The unique length of the twisted wire to each electrode board can be a convenient guide for fitment of the electrodes and wiring assembly to the electrode headset molding.

In another implementation, the signal acquisition system can use a wireless link between the electrodes and the electronic circuitry. Additionally and/or alternatively, the electronic circuitry can be wirelessly linked to an external processor.

Alternative Implementations

It should also be understood that the electrode circuit arrangement, electrodes and electrode headset arrangements described herein can be used in connection in a wide variety of applications outside the implementations described herein. For example the electrode headset arrangement described herein can be used with other known electrode arrangements. Moreover the electrode arrangements described herein can be used to detect other types of bioelectric potentials on parts of the body other than the head, e.g. ECG. The electrodes described herein can also be useful for non-human applications.

It will be understood that the subject matter disclosed in this specification extends to all alternative combinations of two or more of the individual features mentioned or evident from the text or drawings. All of these different combinations constitute various alternative aspects.

A number of embodiments of the invention have been described. Nevertheless, it will be understood that various modifications may be made without departing from the spirit and scope of the invention. For example, the electrode positions shown in the electrode placement scheme 350 in FIG. 4 are approximate and the scheme 350 shown is but one example. Electrode placement schemes with more or fewer electrodes in different positions can be used. If a different electrode placement scheme is desired, the electrode mounts included in the various configurations of electrode headsets described can be positioned differently according to the different electrode placement scheme. Additionally, if required to satisfy a different electrode placement scheme, the bands forming the electrode headset can have different dimensions and/or configurations than shown in the implementations illustrated.

Accordingly, other embodiments are within the scope of the following claims.

What is claimed is:

1. An electrode headset comprising:
a plurality of rigid bands, where the plurality of rigid bands are formed from a material including at least enough flexibility to flex in response to the electrode headset being positioned on a subject's head such that the plurality of rigid bands embrace the subject's head; and
one or more electrode mounts included within the plurality of rigid bands, where each electrode mount is configured to mount an electrode and when the electrode headset is positioned on the subject's head, one or more electrodes mounted therein are positioned according to a desired electrode placement scheme relative to the subject's head.

2. The electrode headset of claim 1, further comprising:
a plurality of voids formed between the plurality of rigid bands, such that portions of the top of the subject's head are exposed when the subject is wearing the electrode headset.

3. The electrode headset of claim 1, wherein each of the one or more electrode mounts comprises an aperture formed within one of the rigid bands, the aperture configured to receive and securely hold an electrode in position against the subject's head.

4. The electrode headset of claim 1, further comprising:
electronic circuitry configured to:
electrically connect to each of the one or more electrodes mounted within the electrode mounts;
receive signals from the one or more electrodes; and
provide an output signal to a processor.

5. The electrode headset of claim 4, wherein the electronic circuitry is embedded within one of the plurality of rigid bands.

6. The electrode headset of claim 4, wherein the electronic circuitry is mounted on one of the plurality of rigid bands.

7. An electrode headset comprising:
a plurality of rigid bands; and
one or more electrode mounts included within the plurality of rigid bands;
wherein the plurality of rigid bands are formed from a material with sufficient flexibility to flex in response to the electrode headset being positioned on a subject's head and sufficient resilience such that when the electrode headset is positioned on the subject's head and electrodes are mounted in the electrode mounts the plurality of rigid bands press the electrodes against the subject's head.

8. The electrode headset of claim 7, wherein the plurality of rigid bands include a first band to position on a first side of the subject's head and a second band to position on a second side of the subject's head and the headset is configured to urge the first band and the second band toward each other.

9. The electrode headset of claim 8, wherein the first band comprises a left temporal band to extend from a back portion of the subject's head toward the subject's left temporal region and the second band comprises a right temporal band to extend from a back portion of the subject's head toward the subject's right temporal region.

10. The electrode headset of claim 8, wherein:
the plurality of rigid bands further includes a center band to position along a central back portion of the subject's head; and
each of the first and second bands comprise a dorsal band extending from the back portion of the subject's head toward opposite sides of the subject's forehead.

11. The electrode headset of claim 7, further comprising:
a plurality of voids formed between the plurality of rigid bands, such that portions of the top of the subject's head are exposed when the subject is wearing the electrode headset.

12. The electrode headset of claim 7, wherein each of the one or more electrode mounts comprises an aperture formed within one of the rigid bands, the aperture configured to receive and securely hold an electrode in position against the subject's head.

13. The electrode headset of claim 7, further comprising:
electronic circuitry configured to:
electrically connect to each of the one or more electrodes mounted within the electrode mounts;
receive signals from the one or more electrodes; and
provide an output signal to a processor.

14. The electrode headset of claim 13, wherein the electronic circuitry is embedded within one of the plurality of rigid bands.

15. An electrode headset comprising:
a plurality of rigid bands including;
a center band configured to position along a central back portion of a subject's head;
a left dorsal band and a right dorsal band, where each dorsal band projects from the center band and extends from the back portion of the subject's head toward the subject's forehead;
a left temporal band and a right temporal band, where each temporal band projects from the center band and extends from the back portion of the subject's head toward the subject's left and right temporal regions respectively;
a plurality of electrode mounts formed within the plurality of rigid bands, where each electrode mount is configured to receive and mount an electrode and when the electrode headset is positioned on the subject's head, one or more electrodes mounted therein are positioned according to a desired electrode placement scheme relative to the subject's head.

16. An electrode headset comprising:
a plurality of rigid bands including;
a center band configured to position along a central back portion of a subject's head, the center band comprising a middle portion positioned between a lower portion extending toward the lower rear of the subject's head and a forked upper portion extending toward the upper back of the subject's head and substantially symmetrical about a sagittal plane;
a left temporal band and a right temporal band, where each temporal band projects from the center band and extends from the rear of the subject's head toward the subject's left and right temporal regions respectively, where each temporal band includes at least a first and a second finger extending from the distal end of the temporal band and where the first and second fingers include distal ends terminating in the frontal region of the subject's head; and
a plurality of electrode mounts formed within each of the center, left temporal and right temporal bands, where each electrode mount is configured to receive and mount an electrode and when the electrode headset is positioned on the subject's head, one or more electrodes mounted therein are positioned according to a desired electrode placement scheme relative to the subject's head.

17. The electrode headset of claim 16, wherein the second finger of each of the left and right temporal bands is forked at the distal end providing two distal termination points and where each distal termination point includes an electrode mount.

18. The electrode headset of claim 16, further comprising:
a chin strap extending from the left temporal band to the right temporal band and configured to position beneath a chin of the subject during use.

19. An electrode headset comprising:
a plurality of bands, where:
the plurality of bands are formed from a soft and stretchable material conformable to a subject's head such that the plurality of bands embrace the subject's head; and
a plurality of voids are formed between the plurality of bands, such that portions of the top of the subject's head remain exposed when the subject is wearing the electrode headset; and
one or more electrode mounts included within the plurality of bands, where each electrode mount is configured to mount an electrode and when the electrode headset is positioned on the subject's head, one or more electrodes mounted therein are positioned according to a desired electrode placement scheme relative to the subject's head.

20. The electrode headset of claim 19, wherein each of the one or more electrode mounts comprises an aperture formed within one of the bands, the aperture configured to receive and securely hold an electrode in position against the subject's head.

21. The electrode headset of claim 19, wherein each of the one or more electrode mounts comprises a pocket formed within one of the bands, the pocket configured to receive and securely hold an electrode in position against the subject's head.

22. The electrode headset of claim 19, further comprising:
electronic circuitry configured to:
electrically connect to each of the one or more electrodes mounted within the electrode mounts;
receive signals from the one or more electrodes; and
provide an output signal to a processor.
